(19) United States
(12) Patent Application Publication
(10) Pub. No.: US 2010/0197749 A1
(43) Pub. Date: Aug. 5, 2010

(54) CHEMICAL COMPOUNDS

(75) Inventors: Scott Cowen, Waltham, MA (US); Tracey Deegan, Waltham, MA (US); Gurmit Grewal, Waltham, MA (US); Vibha Oza, Waltham, MA (US); Jamal Carlos Saeh, Waltham, MA (US); Qibin Su, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (unknown)

(21) Appl. No.: 12/160,979

(22) PCT Filed: Jan. 15, 2007
(86) PCT No.: PCT/GB2007/000090

§ 371 Date: Jul. 15, 2008

Related U.S. Application Data
(60) Provisional application No. 60/759,438, filed on Jan. 17, 2006.

(51) Int. Cl. A61K 31/4196; C07D 249/08; C07D 271/10; A61K 31/4439; C07D 401/12

(52) U.S. Cl. 514/383; 548/267.2; 548/143; 514/340; 546/268.4

(57) ABSTRACT

The invention relates to chemical compounds of formula (I) and (II):

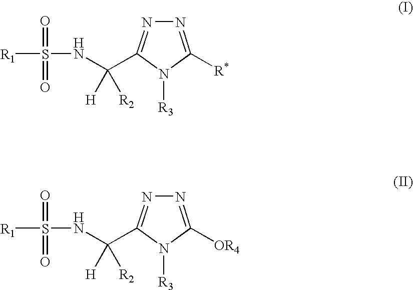

or pharmaceutically acceptable salts thereof, which possess Edg-1 antagonistic activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

### BACKGROUND OF THE INVENTION

EDG (endothelial differentiation gene) receptors belong to a family of closely related, lipid activated G-protein coupled receptors. EDG-1, EDG-3, EDG-5, EDG-6, and EDG-8 (also known as S1P1, S1P3, S1P2, S1P4, and S1 P5) are identified as receptors specific for sphingosine-1-phosphate (SIP). EDG2, EDG4, and EDG7 (known also as LPA1, LPA2, and LPA3, respectively) are receptors specific for lysophosphatidic (LPA). Among the SIP receptor isotypes, EDG-1, EDG-3 and EDG-5 are widely expressed in various tissues, whereas the expression of EDG-6 is confined largely to lymphoid tissues and platelets, and that of EDG-8 to the central nervous system.

EDG receptors are responsible for signal transduction and are thought to play an important role in cell processes involving cell development, proliferation, maintenance, migration, differentiation, plasticity and apoptosis. Certain EDG receptors are associated with diseases mediated by the de novo or deregulated formation of vessels—for example, for diseases caused by ocular neovascularisation, especially retinopathies (diabetic retinopathy, age-related macular degeneration); psoriasis; haemangioblastomas such as “strawberry-marks”; various inflammatory diseases, such as arthritis, especially rheumatoid arthritis, arterial atherosclerosis and atherosclerosis occurring after transplants, endometriosis or chronic asthma; and tumor diseases. EDG receptors are also associated with various inflammatory diseases, such as arthritis, especially rheumatoid arthritis, arterial atherosclerosis and atherosclerosis occurring after transplants, endometriosis or chronic asthma; and, especially, tumor diseases or by lymphocyte interactions, for example, in transplantation rejection, autoimmune diseases, inflammatory diseases, infectious diseases and cancer. An alteration in EDG receptor activity contributes to the pathology and/or symptomology of these diseases. Accordingly, molecules that themselves alter the activity of EDG receptors are useful as therapeutic agents in the treatment of such diseases.

### SUMMARY OF THE INVENTION

In accordance with the present invention, the applicants have hereby discovered novel compounds that are Edg-1 antagonists. These compounds of the present invention provide a method for treating a variety of angiogenesis-related diseases that may be characterized by any abnormal, undesirable or pathological angiogenesis, for example, tumor-related angiogenesis. The compounds may be used to produce an anti-cancer effect mediated alone or in part by antagonism of Edg-1. Such a compound of the invention is expected to possess a wide range of activity in angiogenesis-related diseases including, but not limited to, non-solid tumors such as leukemia, multiple myeloma, hematologic malignancies or lymphoma, and also solid tumors and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors. The compounds of the invention are accordingly useful for their anti-angiogenic (such as anti-cancer) activity and are therefore useful in methods of treatment of the human or animal body.

The invention also relates to processes for the manufacture of said compounds, to pharmaceutical compositions containing them and to their use in the manufacture of medicaments for use in the production of an anti-cancer effect, for example an anti-proliferative effect, in warm-blooded animals such as man.

The present invention includes pharmaceutically acceptable salts of such compounds. Also in accordance with the present invention applicants provide pharmaceutical compositions and a method to use such compounds in the treatment of cancer.

### DETAILED DESCRIPTION OF THE INVENTION

Accordingly, the present invention provides a compound of formula (I)

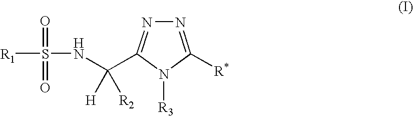

or a pharmaceutically acceptable salt thereof, wherein

R₁ is aryl, heteroaryl, C₁₋₆alkyl, ar(C₁₋₆alkyl), or heteroar(C₁₋₆alkyl) wherein R₁ may be optionally substituted on carbon by one or more substituents selected from C₁₋₃alkyl, halo C₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₂ is C₁₋₆alkyl, C₂₋₆alkenyl, C₂₋₆alkynyl, C₃₋₆cycloalkyl, C₃₋₆cycloalkyl(C₁₋₆alkyl), aryl, heteroaryl, ar(C₁₋₆alkyl), or heteroar(C₁₋₆alkyl) wherein R₂ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₃ is C₁₋₆alkyl, C₃₋₆cycloalkyl, C₃₋₆cycloalkyl(C₁₋₆alkyl), heterocyclylC₁₋₆alkyl, ar(C₁₋₆alkyl), C₃₋₆alkenyl, C₃₋₆alkynyl, or heteroar(C₁₋₆alkyl) wherein R₃ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R* is H, C₁₋₆alkyl, C₃₋₆cycloalkyl, aryl, C-linked heteroaryl, C-linked heterocyclyl, C₃₋₆alkenyl, C₃₋₆alkynyl, ar(C₁₋₆alkyl), heteroar(C₁₋₆alkyl), cycloalkyl(C₁₋₆alkyl), heterocyclyl(C₁₋₆alkyl), acyl, C₁₋₆alkoxycarbonyl(C₁₋₆alkyl), cyano or cyanoalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said C-linked heteroaryl, C-linked heterocyclyl, heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl provided the compound is not N-[1-(4-propyl-4H-1,2,4-triazol-3-yl)ethyl]benzenesulfonamide.

In another embodiment the invention is directed to a compound of formula (II)

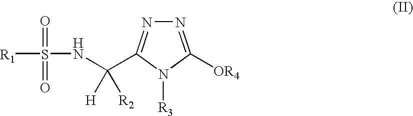

or a pharmaceutically acceptable salt thereof, wherein

R₁ is aryl, heteroaryl, C₁₋₆alkyl, ar(C₁₋₆alkyl), or heteroar(C₁₋₆alkyl) wherein R₁ may be optionally substituted on carbon by one or more substituents selected from C₁₋₃alkyl, halo C₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₂ is heteroar(C₁₋₆alkyl) wherein R₂ may be optionally substituted on carbon by one or more substituents selected from C₁₋₃alkyl, halo C₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₃ is C₁₋₆alkyl, C₃₋₆cycloalkyl, C₃₋₆cycloalkyl(C₁₋₆alkyl), heterocyclylC₁₋₆alkyl, ar(C₁₋₆alkyl), C₃₋₆alkenyl, C₃₋₆alkynyl, or heteroar(C₁₋₆alkyl) wherein R₃ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; and

R₄ is H, C₁₋₆alkyl, C₃₋₆cycloalkyl, aryl, heteroaryl, heterocyclyl, C₃₋₆alkenyl, C₃₋₆alkynyl, ar(C₁₋₆alkyl), heteroar(C₁₋₆alkyl), C₃₋₆cycloalkyl(C₁₋₆alkyl), heterocyclyl(C₁₋₆alkyl), acyl, acyloxy, acylamino, C₁₋₆alkoxycarbonyl(C₁₋₆alkyl), cyano or cyano(C₁₋₆alkyl) wherein R₄ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl, heterocyclyl, heteroar(C₁₋₆alkyl), C₃₋₆cycloalkyl(C₁₋₆alkyl) or heterocyclyl(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl.

In another embodiment the invention is directed to a compound of formula (Ia)

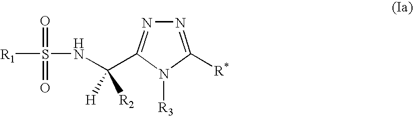

or a pharmaceutically acceptable salt thereof, wherein the definitions of R₁, R₂, R₃ and R* are as defined for formula (I), above.

In a still further embodiment the invention is directed to a compound of formula (IIa)

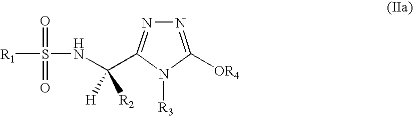

or a pharmaceutically acceptable salt thereof, wherein the definitions of R₁, R₂, R₃ and R₄ are as defined for formula (II), above.

A further aspect of the invention are compounds of formula (3)

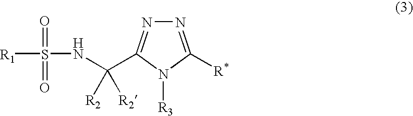

or a pharmaceutically acceptable salt thereof, wherein:

R₁ is aryl, heteroaryl, (C₁-C₆)alkyl, aralkyl, or heteroaralkyl;

R₂ is H, (C₁-C₆)alkyl, aryl, heteroaryl, aralkyl, or heteroaralkyl;

R_{2′} is H or (C₁-C₆)alkyl;

R₃ is (C₁-C₆)alkyl, cycloalkylalkyl, heterocyclyl, aralkyl, (C₃-C₆)alkenyl, (C₃-C₆)alkynyl, or heteroaralkyl;

R* is (C₁-C₆)alkyl, aryl, heteroaryl, heterocyclyl, (C₃-C₆)alkenyl, (C₃-C₆)alkynyl, aralkyl, heteroaralkyl, cycloalkylalkyl, heterocyclylalkyl, acyl, acyloxy, acylamino, or (C₁-C₆)alkoxycarbonyl(C₁-C₆)alkyl, cyano, cyanoalkyl; or

R* is X—R₅, wherein X is S, O, or NR₆, wherein R₆ is H or (C₁-C₆)alkyl; and R₅ is H, (C₁-C₆)alkyl, aryl, heteroaryl, heterocyclyl, (C₃-C₆)alkenyl, (C₃-C₆)alkynyl, aralkyl, heteroaralkyl, cycloalkylalkyl, heterocyclylalkyl, acyl, acyloxy, acylamino, (C₁-C₆)alkoxycarbonyl(C₁-C₆)alkyl, cyano, or cyanoalkyl;

wherein R₁, R₂, R_{2′}, R₃ and R* may be optionally substituted on carbon by one or more substituents selected from (C₁-C₃)alkyl, (C₁-C₃)haloalkyl, (C₁-C₃)alkoxy, alkylthio, —O(CH₂)₁₋₅CF₃, halo, nitro, cyano, ═O, ═S, —OH, —SH, —CF₃, —OCF₃, —CO₂H, CO₂C₁-C₆alkyl, —NH₂, —NHC₁-C₆alkyl, —CONR′R″, or —N(C₁-C₆alkyl)₂ where R′ and R″ are independently alkyl or aryl, or together with the nitrogen to which they are attached form a 4- to 7-membered ring.

What is also provided is a compound of formula 3a or 3b

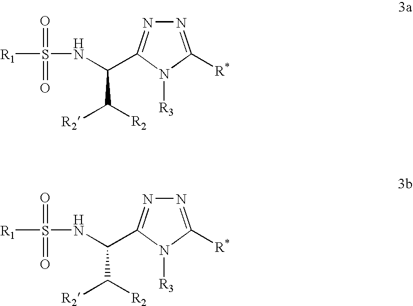

or a pharmaceutically acceptable salt thereof, wherein:

R₁ is aryl, heteroaryl, (C₁-C₆)alkyl, aralkyl, or heteroaralkyl;

R₂ is H, (C₁-C₆)alkyl, aryl, heteroaryl, aralkyl, or heteroaralkyl;

R_{2′} is H or (C₁-C₆)alkyl;

R₃ is (C₁-C₆)alkyl, cycloalkylalkyl, heterocyclyl, aralkyl, (C₃-C₆)alkenyl, (C₃-C₆)alkynyl, or heteroaralkyl;

R* is (C₁-C₆)alkyl, aryl, heteroaryl, heterocyclyl, (C₃-C₆)alkenyl, (C₃-C₆)alkynyl, aralkyl, heteroaralkyl, cycloalkylalkyl, heterocyclylalkyl, acyl, acyloxy, acylamino, (C₁-C₆)alkoxycarbonyl(C₁-C₆)alkyl, cyano, or cyanoalkyl; or

R* is X—R₅, wherein X is S, O, or NR₆, wherein R₆ is H or (C₁-C₆)alkyl; and R₅ is H, (C₁-C₆)alkyl, aryl, heteroaryl, heterocyclyl, (C₃-C₆)alkenyl, (C₃-C₆)alkynyl, aralkyl, heteroaralkyl, cycloalkylalkyl, heterocyclylalkyl, acyl, acyloxy, acylamino, (C₁-C₆)alkoxycarbonyl(C₁-C₆)alkyl, cyano, or cyanoalkyl;

wherein R₁, R₂, R_{2′}, R₃ and R* may be optionally substituted on carbon by one or more substituents selected from (C₁-C₃)alkyl, (C₁-C₃)haloalkyl, (C₁-C₃)alkoxy, alkylthio, —O(CH₂)₁₋₅CF₃, halo, nitro, cyano, ═O, ═S, —OH, SH, CF₃, OCF₃, CO₂H, CO₂C₁-C₆alkyl, NH₂, NHC₁-C₆alkyl, CONR′R″, or —N(C₁-C₆alkyl)₂ where R′ and R″ are independently alkyl or aryl, or together with the nitrogen to which they are attached form a 4- to 7-membered ring.

Unless otherwise stated, the following terms used in the specification and claims have the following meanings.

“Halo” means fluoro, chloro, bromo or iodo.

“(C₁-C₆)Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, tert-butyl, sec-butyl, n-pentyl, n-hexyl, and the like.

An “alkylene,” “alkenylene,” or “alkynylene” group is an alkyl, alkenyl, or alkynyl group, respectively, that is positioned between and serves to connect two other chemical groups. Thus, “(C₁-C₆)alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like.

“(C₂-C₆)Alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(C₂-C₆)Alkynylene” means a linear divalent hydrocarbon radical of two to six carbon atoms or a branched divalent hydrocarbon radical of three to six carbon atoms, containing at least one triple bond, for example, as in ethynylene, propynylene, and the like. “(C₃-C₆)Cycloalkyl” means a hydrocarbon ring containing from 3 to 6 carbon atoms for example, cyclopropyl, cyclobutyl, cyclopentyl, cyclohexyl. Where possible, the cycloalkyl group may contain double bonds, for example, 3-cyclohexen-1-yl. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

“(C₃-C₆)Cycloalkyl(C₁-C₆)alkyl” means a (C₃-C₆)cycloalkyl group covalently attached to a (C₁-C₆)alkylene group, both of which are defined herein. The term “heterocyclyl” means a monocyclic, fused, bridged, or spiro bicyclic heterocyclic ring system(s) which may be saturated or partially unsaturated and which optionally may be substituted with up to 4 groups selected from those recited above as substituents for alkyl. Monocyclic heterocyclic rings contain from about 3 to 12 ring atoms, with from 1 to 5 heteroatoms selected from N, O, and S, and preferably from 3 to 7 member atoms, in the ring. Bicyclic heterocycles contain from 7 to 17 member atoms, preferably 7 to 12 member atoms, in the ring. Bicyclic heterocycles contain from about 7 to about 17 ring atoms, preferably from 7 to 12 ring atoms. Bicyclic heterocyclic(s) rings may be fused, spiro, or bridged ring systems. Examples of heterocyclic groups include cyclic ethers (oxiranes) such as ethyleneoxide, tetrahydrofuran, dioxane, and substituted cyclic ethers. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiepin-4-yl. Other commonly employed heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

“C-linked heterocyclyl” means that the heterocyclyl group is attached to the triazole ring via a carbon atom of the heterocyclyl group.

“Heterocyclyl(C₁-C₆)alkyl” means a heterocyclyl group covalently attached to a (C₁-C₆)alkylene group, both of which are defined herein. The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, 4-trifluoromethyl and the like.

Ar(C₁-C₆)alkyl means an aryl group covalently attached to a (C₁-C₆)alkylene group, both of which are defined herein. Examples of aralkyl groups include benzyl, phenylethyl, 3-(3-chlorophenyl)-2-methylpentyl, and the like

The term “heteroaryl” means an aromatic mono-, bi-, or polycyclic ring incorporating one or more (i.e. 1-4) heteroatoms selected from N, O, and S. The term heteroaryl includes both monovalent species and divalent species. Examples of monocyclic heteroaryl include, but are not limited to substituted or unsubstituted thienyl, furanyl, pyrrolyl, imidazolyl, pyrazolyl, isothiazolyl, isoxazolyl, triazolyl, tetrazolyl, pyridinyl, pyrazinyl, pyrimidinyl, piperidinyl, pyrrolidinyl, piperazinyl, azetidinyl, aziridinyl, morpholinyl, thietanyl, oxetaryl. Monocyclic diheterocycles include, but are not limited to, 1-, 2-, 4-, or 5-imidazolyl, 1-, 3-, 4-, or 5-pyrazolyl, 3-, 4-, or 5-isothiazolyl, 3-, 4-, or 5-isoxazolyl, 1,3-, or 5-triazolyl, 1-, 2-, or 3-tetrazolyl, 2-pyrazinyl, 2-, 4-, or 5-pyrimidinyl, 1- or 2-piperazinyl, 2-, 3-, or 4-morpholinyl. Examples of bicyclic and polyclic heteroaryl groups include, but are not limited to include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinolinyl, 1-, 3-, 4-, 5-, 6-, 7-, or 8-isoquinolinyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4-aH carbazolyl, 1-, 2-, 3-, 4-, 5-, 6-, 7-, or 8-carbazolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoquinolinyl, 2-, 3-, 4, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 5-4H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 7-, 8-, 9-, 10, or 1′-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 1′-1H-pyrrolo[1,2-b][2]benzazapinyl. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

“C-linked heteroaryl” means that the heteroaryl group is attached to the triazole ring via a carbon atom of the heteroaryl group.

“Heteroar(C₁-C₆alkyl)” means an heteroaryl group covalently attached to a (C₁-C₆)alkylene group, both of which are defined herein. Examples of heteroaralkyl groups include pyridin-3-ylmethyl, 3-(benzofuran-2-yl)propyl, and the like.

“Haloalkyl” means alkyl substituted with one or more same or different halo atoms, e.g., —CH₂Cl, —CF₃, —CH₂CF₃, —CH₂CCl₃, and the like.

“Optionally substituted” means that the group at issue is optionally substituted as provided.

Some compounds of the formula (I) may have chiral centres and/or geometric isomeric centres (E- and Z-isomers), and it is to be understood that the invention encompasses all such optical, diastereoisomers and geometric isomers that possess Edg-1 antagonistic activity.

The invention relates to any and all tautomeric forms of the compounds of the formula (I) that possess Edg-1 antagonistic activity.

It is also to be understood that certain compounds of the formula (I) can exist in solvated as well as unsolvated forms such as, for example, hydrated forms. It is to be understood that the invention encompasses all such solvated forms which possess Edg-1 antagonistic activity.

Particular values of variable groups are as follows. Such values may be used where appropriate with any of the definitions, claims or embodiments defined hereinbefore or hereinafter unless specifically indicated otherwise.

R₁ is aryl wherein aryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₁ is heteroaryl wherein heteroaryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl.

R₁ is aryl wherein aryl may be optionally substituted on carbon by one or more halo.

R₁ is phenyl wherein phenyl may be optionally substituted on carbon by one or more substituents selected from C₁₋₃ alkyl, haloC₁₋₃ alkyl, C₃₋₆cycloalkyl, C₁₋₃ alkoxy, C₁₋₃ alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring,

R₁ is phenyl wherein phenyl may be optionally substituted on carbon by one or more halo.

R₁ is phenyl wherein phenyl may be optionally substituted on carbon by chloro. R₁ is p-chlorophenyl.

For compounds of formula (I) and (Ia) values for R₂ are as follows:

R₂ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₂ is C₁₋₆alkyl.

R₂ is C₁₋₆alkyl, C₂₋₆alkenyl, C₂₋₆alkynyl, C₃₋₆cycloalkyl(C₁₋₆alkyl), ar(C₁₋₆alkyl), or heteroar(C₁₋₆alkyl) wherein R₂ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl.

R₂ is C₁₋₆alkyl, C₂₋₆alkenyl, C₂₋₆alkynyl, C₃₋₆cycloalkyl(C₁₋₆alkyl), or heteroar(C₁₋₆alkyl).

R₂ is C₂₋₆alkenyl wherein C₂₋₆alkenyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₂ is C₂₋₆alkenyl.

R₂ is C₃₋₆cycloalkyl(C₁₋₆alkyl) wherein C₃₋₆cycloalkyl(C₁₋₆alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₂ is C₃₋₆cycloalkyl(C₁₋₆alkyl).

R₂ is ar(C₁₋₆alkyl) wherein ar(C₁₋₆alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₂ is ar(C₁₋₆alkyl).

R₂ is benzyl wherein benzyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₂ is benzyl optionally substituted by halo.

R₂ is p-fluorobenzyl.

R₂ is benzyl.

R₂ is selected from methyl, 2-propyl, 2-propenyl, benzyl, p-fluorobenzyl and cyclopropylmethyl.

The following values for R₂ may be used where appropriate with any of the definitions, claims or embodiments defined hereinbefore or hereinafter.

R₂ is heteroar(C₁₋₆alkyl) wherein heteroar(C₁₋₆alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆ alkyl)carbamoyl, benzyl, benzyloxycarbonyl, benzoyl and phenylsulphonyl.

R₂ is heteroar(C₁₋₃alkyl) wherein heteroar(C₁₋₃alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring wherein if said heteroar(C₁₋₃alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl

R₂ is pyridinylmethyl wherein pyridinylmethyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₂ is pyridinylmethyl wherein pyridinylmethyl may be optionally substituted on carbon by halo.

R₂ is selected from pyridin-2-ylmethyl, pyridin-3-ylmethyl and 4-fluoropyridin-2-ylmethyl.

R₃ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₃ is C₁₋₃alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₃ is C₁₋₆alkyl.

R₃ is C₁₋₃alkyl.

R₃ is selected from methyl, ethyl, and isopropyl.

R₃ is ethyl.

R* is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R* is C₁₋₆alkyl or C₁₋₃cycloalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R* is H, C₁₋₆alkyl or C₁₋₃cycloalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R* is C₁₋₆alkyl, C₃₋₆alkenyl, C₃₋₆alkynyl, ar(C₁₋₆alkyl), heteroar(C₁₋₆alkyl), cycloalkyl(C₁₋₆alkyl), heterocyclyl(C₁₋₆alkyl), C₁₋₆alkoxycarbonyl(C₁₋₆alkyl) or cyanoalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl.

R* is C-linked heteroaryl or C-linked heterocyclyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said C-linked heteroaryl or C-linked heterocyclyl contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl.

R* is aryl wherein aryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R* is acyl wherein acyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₄ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₄ is C₁₋₃alkyl wherein C₁₋₃alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

R₄ is C₁₋₆alkyl.

R₄ is C₁₋₃alkyl.

R₄ is methyl.

In a further aspect of the invention there is provided a compound of formula (I) or Formula (Ia) or a pharmaceutically acceptable salt thereof, wherein

R₁ is aryl wherein aryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring;

R₂ is C₁₋₆alkyl, C₃₋₆cycloalkyl(C₁₋₆alkyl), ar(C₁₋₆alkyl), or heteroar(C₁₋₆alkyl) wherein R₂ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₃ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring; and

R* is H, C₁₋₆alkyl or C₃₋₆cycloalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

In a further aspect of the invention there is provided a compound of formula (I) or Formula (Ia) or a pharmaceutically acceptable salt thereof, wherein

R₁ is phenyl wherein phenyl may be optionally substituted on carbon by one or more substituents selected from C₁₋₃alkyl, haloC₁₋₃alkyl or halo;

R₂ is C₁₋₆alkyl, C₂₋₆alkenyl, C₃₋₆cycloalkyl(C₁₋₆alkyl), ar(C₁₋₆alkyl), or heteroar(C₁₋₆alkyl) wherein R₂ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl,

C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₃ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring; and

R* is H, C₁₋₆alkyl or C₃₋₆cycloalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

In a further aspect of the invention there is provided a compound of formula (II) or Formula (IIa) or a pharmaceutically acceptable salt thereof, wherein

R₁ is aryl wherein aryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and W′ are independently C₁₋₆alkyl or aryl, or together with the nitrogen to which they are attached form a 4- to 7-membered ring;

R₂ is heteroar(C₁₋₆alkyl) wherein heteroar(C₁₋₆alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₃ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring; and

R₄ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

In a further aspect of the invention there is provided a compound of formula (II) or Formula (IIa) or a pharmaceutically acceptable salt thereof, wherein

R₁ is phenyl wherein phenyl may be optionally substituted on carbon by one or more substituents selected from C₁₋₃alkyl, haloC₁₋₃alkyl or halo;

R₂ is heteroar(C₁₋₃alkyl) wherein heteroar(C₁₋₃alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R₃ is C₁₋₃alkyl wherein C₁₋₃alkyl may be optionally substituted on carbon by one or more substituents selected from C₁₋₃ alkyl, haloC₁₋₃ alkyl, C₃₋₆cycloalkyl, C₁₋₃ alkoxy, C₁₋₃ alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring; and

R₄ is C₁₋₃alkyl wherein C₁₋₃alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

Another aspect of the invention is any one of Examples 1-17 or a pharmaceutically acceptable salt thereof.

A further aspect of the invention is any one of Examples 11, 12 or 14 or a pharmaceutically acceptable salt thereof.

A still further aspect of the invention is any one of Examples 15, 16 or 17 or a pharmaceutically acceptable salt thereof.

Another aspect of the present invention provides processes a and b for preparing compounds of formula (I) and (Ia) or a pharmaceutically acceptable salt or an in vivo hydrolyzable ester thereof which processes (wherein variable groups are, unless otherwise specified, as defined in formula (I) and (II)) comprise the following:

Process a) reacting a compound of formula (1e)

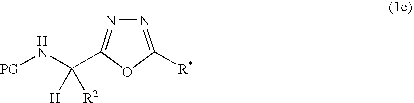

with an amine of the formula (2)

R₃—NH₂ (2)

wherein PG is R₁SO₂ or a protecting group, e,g, BOC

and, if PG is a protecting group, further reacting with R₁SO₂Cl

process b) reacting a compound of formula (1e′)

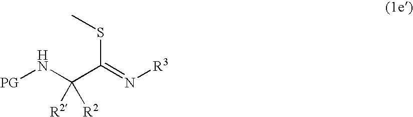

with a hydrazide of formula (2a)

R*—CONHNH₂ (2a)

and, if PG is a protecting group, further reacting with R₁SO₂Cl

and thereafter if necessary:
i) converting a compound of the formula (I) or (Ia) into another compound of the formula (I) or (Ia);
ii) removing any protecting groups;
iii) forming a pharmaceutically acceptable salt.

Another aspect of the present invention provides process c for preparing compounds of formula (II) and (IIa) or a pharmaceutically acceptable salt or an in vivo hydrolyzable ester thereof which processes (wherein variable groups are, unless otherwise specified, as defined in formula (II) comprises the following:

Process c) reacting a compound of formula (2f)

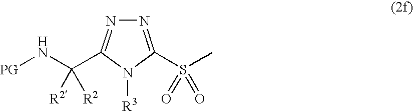

with a compound of the formula (2g)

R⁴ONa (2g)

wherein PG is R₁SO₂

and thereafter if necessary:
i) converting a compound of the formula (II) or (IIa) into another compound of the formula (II) or (IIa);
ii) removing any protecting groups;
iii) forming a pharmaceutically acceptable salt.

Specific reaction conditions for the above reactions are as follows:

Process a) Compounds of formula 1e and R—NH₂ are reacted together in the presence of a suitable solvent such as ethanol or THF.

Process b) Compounds of formula (1e′) and R*—CONHNH₂ are reacted together in the presence of trifluoroacetic acid and a suitable solvent like THF.

Process c) Compounds of formula 2f and 2g (R⁴O—Na) are reacted together in an appropriate solvent such as THF.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. For example, the carbon to which R₂ is attached in a compound of Formula (I) is an asymmetric center and thus the compound of Formula (I) can exist as an (R)- or (S)-stereoisomer relative to that carbon. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

A “pharmaceutically acceptable counterion” means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate, and the like.
1. A “pharmaceutically acceptable salt” of a compound means a salt that is pharmaceutically acceptable and that possesses the desired pharmacological activity of the parent compound. Such salts include:
   acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or
2. salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Leaving group” has the meaning conventionally associated with it in synthetic organic chemistry i.e., an atom or group capable of being displaced by a nucleophile and includes halo(such as chloro, bromo, iodo), alkanesulfonyloxy (such as mesyloxy or trifluorosulfonyloxy) or arenesulfonyloxy (such as tosyloxy), and the like. Leaving Groups are well known in the art and are catalogued in “Protective Groupsin Organic Synthesis 3^{rd} Ed.”, edited by Theodora Green and Peter Wuts (John Wiley, 1999).

What is also provided is a compound of formula Ia or Ib or a pharmaceutically acceptable salt, prodrug, or solvate thereof in association with a pharmaceutically acceptable carrier, diluent, or excipient.

What is also provided is a compound of formula Ia or Ib or a pharmaceutically acceptable salt, prodrug, or solvate thereof, which is an Edg-1 antagonist useful for controlling pathologically angiogenic diseases, thrombosis, cardiac infarction, coronary heart diseases, arteriosclerosis, tumors, osteoporosis, inflammations or infections.

What is also provided is a method of treating a disease or condition mediated by Edg-1 which comprises administering to a patient in need of such treatment a compound of formula compound of formula Ia or Ib or a pharmaceutically acceptable salt, prodrug, or solvate thereof.

What is also provided is a compound of formula I or a pharmaceutically acceptable salt, prodrug, or solvate thereof, which is an Edg-1 antagonist useful for controlling pathologically angiogenic diseases, thrombosis, cardiac infarction, coronary heart diseases, arteriosclerosis, tumors, osteoporosis, inflammations or infections.

The compounds of Formula (3) may be administered in the form of a pro-drug which is broken down in the human or animal body to give a compound of the Formula (3). A “Pro-drug” is any compound which releases an active parent drug according to Formula (3) in vivo when such prodrug is administered to a mammalian subject. Prodrugs of a compound of Formula (3) are prepared by modifying functional groups present in the compound of Formula (3) in such a way that the modifications may be cleaved in vivo to release the parent compound. Examples of prodrugs include, but are not limited to esters (e.g., acetate, formate, and benzoate derivatives), carbamates (e.g., N,N-dimethylaminocarbonyl) of hydroxy functional groups in compounds of Formula (3), and the like.

Various forms of pro-drugs are known in the art. For examples of such pro-drug derivatives, see:
1. Design of Prodrugs, edited by H. Bundgaard, (Elsevier, 1985) and Methods in Enzymology, Vol. 42, p. 309-396, edited by K. Widder, et al. (Academic Press, 1985);
2. A Textbook of Drug Design and Development, edited by Krogsgaard-Larsen and H. Bundgaard, Chapter 5 “Design and Application of Prodrugs”, by H. Bundgaard p. 113-191 (1991);
3. H. Bundgaard, Advanced Drug Delivery Reviews, 8, 1-38 (1992);
4. H. Bundgaard, et al., Journal of Pharmaceutical Sciences, 77, 285 (1988);
5. N. Kakeya, et al., Chem Pharm Bull, 32, 692 (1984);
6. K. Beaumont et. al., Current Drug Metabolism, 4, 461 (2003).

An in-vivo hydrolysable ester of a compound of the Formula (3) containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the Formula (3) containing a hydroxy group includes inorganic esters such as phosphate esters (including phosphoramidic cyclic esters) and a-acyloxyalkyl ethers and related compounds which as a result of the in-vivo hydrolysis of the ester breakdown to give the parent hydroxy group/s. Examples of a-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

“Treating” or “treatment” of a disease includes:
1. preventing the disease, i.e. causing the clinical symptoms of the disease not to develop in a mammal that may be exposed to or predisposed to the disease but does not yet experience or display symptoms of the disease;
2. inhibiting the disease, i.e., arresting or reducing the development of the disease or its clinical symptoms; or
3. relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

### Preparation of Invention Compounds

Compounds of the invention can be prepared as provided in Schemes 1-3, below. The skilled artisan will recognize that the invention compounds can be prepared from chiral starting materials or via racemic synthesis, followed by chiral separation, to isolate the enantiomers.

### Pharmaceutical Formulations

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of any of the foregoing formula or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

What is also provided is a compound of formula (I), (Ia), (II) or (IIa) or a pharmaceutically acceptable salt, prodrug, or solvate thereof in association with a pharmaceutically acceptable carrier, diluent, or excipient.

According to a further aspect of the present invention there is provided a compound of the formula (I), (Ia), (II) or (IIa) or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their Edg-1 antagonistic properties. Accordingly the compounds of the present invention are expected to be useful in the treatment of diseases or medical conditions mediated alone or in part by Edg-1, i.e. the compounds may be used to produce an Edg-1 antagonistic effect in a warm-blooded animal such as man in need of such treatment.

What is also provided is a method of treating a disease or condition mediated by Edg-1 which comprises administering to a patient in need of such treatment a compound of formula compound of formula (I), (Ia), (II) or (IIa) or a pharmaceutically acceptable salt, prodrug, or solvate thereof.

Thus the compounds of the present invention provide a method for treating cancer characterized by the antagonistic effect of Edg-1, i.e. the compounds may be used to produce an anti-cancer effect mediated alone or in part by the antagonistic effect of Edg-1.

Thus the compounds of the present invention provide a method for treating a variety of angiogenesis-related diseases that may be characterized by any abnormal, undesirable or pathological angiogenesis, for example tumor-related angiogenesis. The compounds may be used to produce an anti-cancer effect mediated alone or in part by antagonism of Edg-1. Such a compound of the invention is expected to possess a wide range of activity in angiogenesis-related diseases including, but not limited to, non-solid tumors such as leukemia, multiple myeloma, hematologic malignancies or lymphoma, and also solid tumors and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors. Thus according to this aspect of the invention there is provided the use of a compound of the formula (I), (Ia), (II) or (IIa) or a pharmaceutically acceptable salt thereof in the manufacture of a medicament for use in the treatment of angiogenesis-related diseases including, but not limited to, non-solid tumors such as leukemia, multiple myeloma, hematologic malignancies or lymphoma, and also solid tumors and their metastases such as melanoma, non-small cell lung cancer, glioma, hepatocellular carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors. In another embodiment the invention is directed to a method of treating of angiogenesis-related diseases including non-solid tumors, solid tumors and their metastases, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors, in a warm-blooded animal in need of such treatment which comprises administering to said animal an effective amount of a compound of formula (I), (Ia), (II) or (IIa) or a pharmaceutically acceptable salt thereof.

Excessive vascular growth also contributes to numerous non-neoplastic disorders for which the compounds of the invention may be useful in treating. These non-neoplastic angiogenesis-related diseases include: atherosclerosis, haemangioma, haemangioendothelioma, angiofibroma, vascular malformations (e.g. Hereditary Hemorrhagic Teleangiectasia (HHT), or Osler-Weber syndrome), warts, pyogenic granulomas, excessive hair growth, Kaposis' sarcoma, scar keloids, allergic oedema, psoriasis, dysfunctional uterine bleeding, follicular cysts, ovarian hyperstimulation, endometriosis, respiratory distress, ascites, peritoneal sclerosis in dialysis patients, adhesion formation result from abdominal surgery, obesity, rheumatoid arthritis, synovitis, osteomyelitis, pannus growth, osteophyte, hemophilic joints, inflammatory and infectious processes (e.g. hepatitis, pneumonia, glomerulonephritis), asthma, nasal polyps, liver regeneration, pulmonary hypertension, retinopathy of prematurity, diabetic retinopathy, age-related macular degeneration, leukomalacia, neovascular glaucoma, corneal graft neovascularization, trachoma, thyroiditis, thyroid enlargement, and lymphoproliferative disorders.

Thus according to this aspect of the invention there is provided a compound of the formula (I), (Ia), (II) or (IIa) or a pharmaceutically acceptable salt thereof, as defined hereinbefore for use as a medicament.

According to a further aspect of the invention there is provided the use of a compound of the formula (I), (Ia), (II) or (IIa) or a pharmaceutically acceptable salt thereof, as defined hereinbefore in the manufacture of a medicament for use in the production of a Edg-1 antagonistic effect in a warm-blooded animal such as man.

According to this aspect of the invention there is provided the use of a compound of the formula (I), (Ia), (II) or (IIa), or a pharmaceutically acceptable salt thereof, as defined hereinbefore in the manufacture of a medicament for use in the production of an anti-cancer effect in a warm-blooded animal such as man.

According to a further feature of the invention, there is provided a compound of the formula (I), (Ia), (II) or (IIa) or a pharmaceutically acceptable salt thereof, as defined herein before in the manufacture of a medicament for use in the treatment of pathologically angiogenic diseases, thrombosis, cardiac infarction, coronary heart diseases, arteriosclerosis, tumors, osteoporosis, inflammations or infections in a warm-blooded animal such as man.

According to a further feature of this aspect of the invention there is provided a method for producing a Edg-1 antagonistic effect in a warm-blooded animal, such as man, in need of such treatment which comprises administering to said animal an effective amount of a compound of formula (I), (Ia), (II) or (IIa) or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), (Ia), (II) or (IIa) or a pharmaceutically acceptable salt thereof, as defined above.

According to an additional feature of this aspect of the invention there is provided a method of treating pathologically angiogenic diseases, thrombosis, cardiac infarction, coronary heart diseases, arteriosclerosis, tumors, osteoporosis, inflammations or infections, in a warm-blooded animal, such as man, in need of such treatment which comprises administering to said animal an effective amount of a compound of formula (I), (Ia), (II) or (IIa) or a pharmaceutically acceptable salt thereof as defined herein before.

In a further aspect of the invention there is provided a pharmaceutical composition which comprises a compound of the formula (I), (Ia), (II) or (IIa) or a pharmaceutically acceptable salt thereof, as defined herein before in association with a pharmaceutically-acceptable diluent or carrier for use in the production of a Edg-1 antagonistic effect in a warm-blooded animal such as man.

In a further aspect of the invention there is provided a pharmaceutical composition which comprises a compound of the formula (I), (Ia), (II) or (IIa) or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), (Ia), (II) or (IIa) or a pharmaceutically acceptable salt thereof, as defined herein before in association with a pharmaceutically-acceptable diluent or carrier for use in the treatment of pathologically angiogenic diseases, thrombosis, cardiac infarction, coronary heart diseases, arteriosclerosis, tumors, osteoporosis, inflammations or infections in a warm-blooded animal such as man.

### Combinations

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:
1. antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
2. cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
3. agents which inhibit cancer cell invasion (for example metalloproteinase inhibitors like marimastat and inhibitors of urokinase plasminogen activator receptor function);
4. inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib,), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
5. antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
6. vascular damaging agents such as Combretastatin A4 and compounds disclosed in International Patent Applications WO 99/02166, WO 00/40529, WO 00/41669, WO 01/92224, WO 02/04434 and WO 02/08213;
7. antisense therapies, for example those which are directed to the targets listed above, such as ISIS 2503, an anti-ras antisense;
8. gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
9. immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Biological Activity

The following assay can be used to measure the effects of the compounds of the present invention as S1P1/Edg1 inhibitors.

### A. In Vitro Cell Based Receptor Activation Assay-Transfluor Assay

This cell-based assay was designed to assess the ability of small molecule antagonists to inhibit activation of the GPCR S1P1 in the presence of its cognate ligand S1P. The assay used technology initially developed by Norak Biosciences (Xsira Pharmaceutical) and presently owned by Molecular Devices. A human osteogenic sarcoma (U20S) cell line overexpressing the EDG-1/S1P1) receptor as well as a beta-arrestin/green fluorescent protein (GFP) construct hereafter termed EDG-1 Transfluor U20S WT Clone #37 was employed.

Using a high content screening approach (Cellomics Arrayscan), receptor activity was measured by assessment of the relocalization of beta-arrestin GFP in response to stimulation of EDG-1 by SIP. Specifically, EDG-1 Transfluor U20S WT Clone #37 cells were plated at a density of 6250 cells in 40 μL medium per well in 384 well plastic bottomed microtiter plates (BD Falcon) and incubated overnight at 37° C./5% CO₂. Prior to screening, compounds were dissolved in 100% dimethyl sulfoxide (DMSO) to a final stock concentration of 10 mM. Compounds were then serially diluted at 30× final concentration in EDG-1 Transfluor cell growth medium containing 30% DMSO using the Tecan Genesis instrument. These 30× plates were then diluted to 6× final concentration with EDG-1 Transfluor growth medium just prior to dosing. Cells were then dosed with 10 μL per well of 6× compound dilutions or 6% DMSO and pre-incubated for 15 minutes at room temperature. Cell plates were dosed with 10 μL per well 6×SIP EDG-1 Transfluor growth medium, then incubated for 45 minutes at 37° C./5% CO₂. Final concentration in the well of DMSO was 1%, compound was 1× (3-fold, 9 point IC₅₀ dilutions starting at 100 μM final concentration), and either 375 nM or 750 nM S1P ligand. Cell plates were then fixed by adding 50 μL per well of 5% formaldehyde in 1× Dulbecco's phosphate buffered saline (DPBS) directly and incubating for 30 minutes at room temperature in darkness. Fixative was removed and replaced with 50 μL per well of 1×DPBS, after which cells were stained with 10 μg/mL final concentration of Hoechst 33342 (Molecular Probes) for 15 minutes at room temperature in darkness. Stain was then removed from the plates and replaced with 50 μL per well of 1×DPBS using the BioTek ExL405 plate washer. Plates were then sealed and analysed on the Cellomics Arrayscan using the GPCR signalling algorithm. EC₅₀ values were then calculated using IDBS ActivityBase software.

In this assay, compounds of the invention exhibit EC₅₀ values <100 μM. For example, the compound of Example 11 exhibited an EC₅₀ value 0.670 μM and the compound of Example 16 exhibited an EC₅₀ value of 0.133 μM.

### EXPERIMENTAL SECTION

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulphate, work-up procedures were carried out using traditional layer separating techniques or an ALLEXIS (MTM) automated liquid handler, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz, a Bruker Dpx400 operating at 400 MHz or a Bruker Advance operating at 500 MHz. The following abbreviations have been used: s, singlet; d, doublet; t, triplet; q, quartet; m, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88)) and acetonitrile;

(x) the following analytical HPLC methods were used; in general, reversed-phase silica was used with a flow rate of about 1 ml per minute and detection was by Electrospray Mass Spectrometry and by UV absorbance at a wavelength of 254 nm; for each method Solvent A was water and Solvent B was acetonitrile; the following columns and solvent mixtures were used:—

Preparative HPLC was performed on C18 reversed-phase silica, on a Phenominex “Gemini” preparative reversed-phase column (5 microns silica, 110A, 21.1 mm diameter, 100 mm length) using decreasingly polar mixtures as eluent, for example decreasingly polar mixtures of water (containing 0.1% formic acid or 0.1% ammonia) as solvent A and acetonitrile as solvent B; either of the following preparative HPLC methods were used:

Method A: a solvent gradient over 9.5 minutes, at 25 mls per minute, from a 85:15 mixture of solvents A and B respectively to a 5:95 mixture of solvents A and B.

Method B: a solvent gradient over 9.5 minutes, at 25 mls per minute, from a 60:40 mixture of solvents A and B respectively to a 5:95 mixture of solvents A and B.

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xii) the following abbreviations have been used:—
DMF N,N-dimethylformamide
DMSO dimethylsulphoxide
EtOAc ethyl acetate
Et₃N triethylamine
CH₂Cl₂ dichloromethane
CH₂I₂ diiodomethane
THF tetrahydrofuran
TFA trifluoroacetic acid
DMA N-dimethylacetamide
DCM dichloromethane
HATU O-(7-azabenzotriazol-1-yl)-N,N,N′,N′-tetramethyluronium hexafluorophosphate
MeCN Acetonitrile
DIPEA/Hunig's base diisopropyl ethylamine
PPh₃ triphenyl phosphine
TFAA trifluoroacetic anhydride
TMSCHN₂ trimethylsilyl diazomethane
HOBt N-hydroxybenzotriazole
EDCI 1-(3-Dimethylaminopropyl)-3-ethyl-carbodiimide hydrochloride
RT or rt room temperature
Boc N-(test-butoxycarbonyl)
Lawesson's reagent 2,4-bis(4-methoxyphenyl)-1,3-dithia-2,4-diphosphatane-2,4-disulfide bearing the following structure

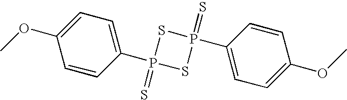

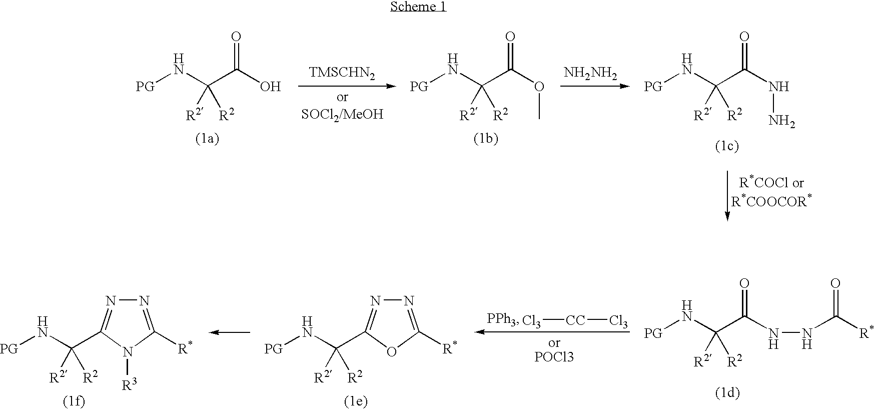

Compounds of formula (I) can also be generated as shown in scheme 2

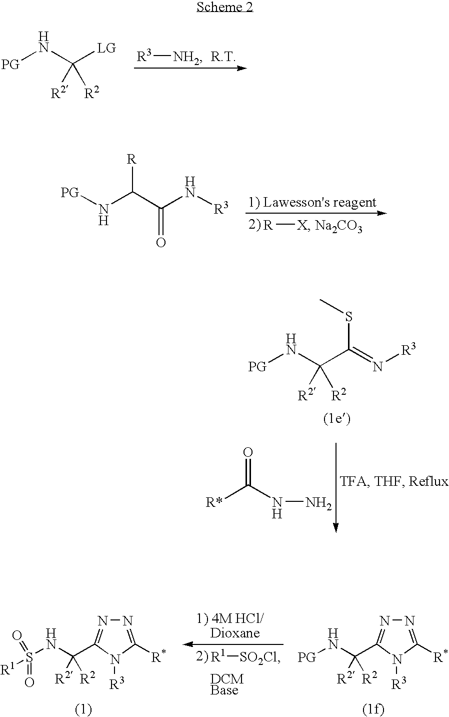

Compounds of formula (II) may be also prepared as shown below in scheme 3

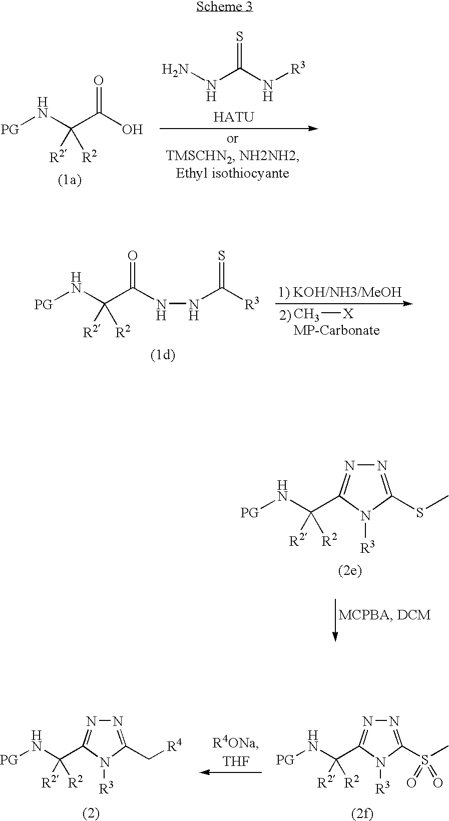

In cases where commercially available (1a) is used, an additional step may be necessary as shown below to convert (1f) to compounds of formula 1

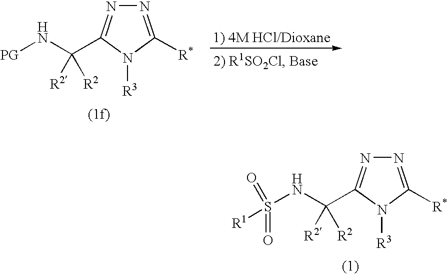

Compound of formula (1a) may be either commercially available or may be generated by reacting the appropriate commercially available amino acid with commercially available sulfonyl chlorides.

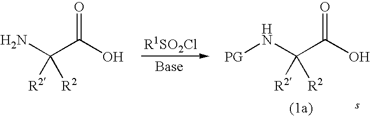

### Example 1

### 4-Chloro-N-[1-(4-methyl-5-methyl-4H-1,2,4-triazol-3-yl)-2-phenylethyl]benzenesulfonamide

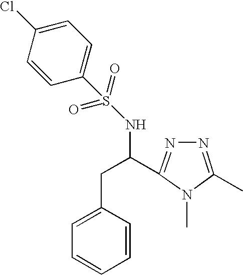

A solution of 4-chloro-N-[1-(5-methyl-1,3,4-oxadiazol-2-yl)-2-phenylethyl]benzenesulfonamide (Intermediate 1a, 50 mg) in methyl amine (33% wt. in EtOH) was heated to 150° C. in a microwave for 1.5 hours. The reaction mixture was cooled and concentrated to yield crude product which was purified using reverse phase HPLC to afford 4-chloro-N-[1-(4-methyl-5-methyl-4H-1,2,4-triazol-3-yl)-2-phenylethyl]benzenesulfonamide (30 mg,). ¹H NMR (300 MHz, DMSO-d6) 8.87 (1H, d), 7.50 (4H, m), 7.14 (5H, m), 4.73 (1H, m), 3.43 (3H, s), 3.12 (1H, dd), 2.98 (1H, dd), 2.32 (3H, s). M/Z=390.

Examples 2-8, shown in Table 1, were prepared in a manner analogous to that described for Example 1 using the intermediate oxadiazole (Intermediate) indicated in TABLE 1 and the appropriate commercially available amine.

TABLE 1

| Ex | Structure | NMR | MS | Intermediate |
|---|---|---|---|---|
| 2 | 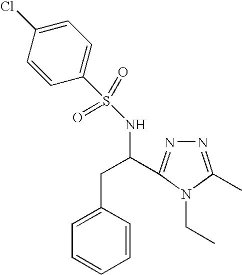 4-chloro-N-[1-(4-ethyl-5- methyl-4H-1,2,4-triazol-3-yl)-2- phenylethyl]benzenesulfonamide | ¹H NMR (300 MHz, CDCl₃) 8.31 (1H, d), 7.68 (2H, m), 7.41 (2H, m), 7.19 (4H, m), 6.95 (1H, d), 4.78 (1H, d), 3.88 (1H, m), 3.66 (1H, m), 3.40 (1H, m), 3.26 (1H, m), 2.48 (3H, s), 0.98 (3H, t) | 405 | 1a |
| 3 | 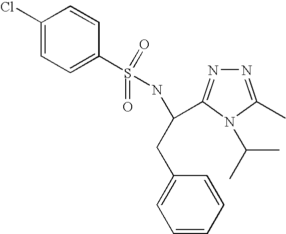 4-chloro-N-[1-(4-isopropyl-5- methyl-4H-1,2,4-triazol-3-yl)-2- phenylethyl]benzenesulfonamide | ¹HNMR (300 MHz, MeOD) 7.57 (2H, d), 7.36 (2H, d), 7.09 (3H, m), 6.94 (2H, m) 4.88 (1H, m), 4.72 (1H, m), 4.03 (2H, m), 3.12 (1H, m), 2.98 (1H, m), 2.64 (3H, s), 1.46 (3H, d), 1.19 (3H, d) | 418 | 1a |
| 4 | 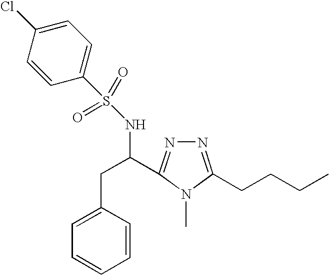 N-[1-(5-butyl-4-methyl-4H- 1,2,4-triazol-3-yl)-2- phenylethyl]-4- chlorobenzenesulfonamide | ¹H NMR (300 MHz, CDCl₃) 8.88 (1H, d), 7.51 (4H, m), 7.18 (5H, m), 4.73 (1H, m), 3.35 (3H, s), 3.10 (1H, m), 2.62 (2H, m), 1.52 (2H, m), 1.30 (2H, m), 0.91 (3H, t) | 433 | 2a |
| 5 | 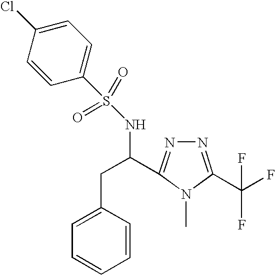 4-chloro-N-{1-[4-methyl-5- (trifluoroinethyl)-4H-1,2,4- triazol-3-yl]-2-phenylethyl}- benzenesulfonamide | ¹H NMR (300 MHz, CDCl₃) 8.99 (1H, d), 7.49 (4H, m), 7.16 (5H, m), 4.77 (1H, m), 3.45 (3H, s), 3.16 (2H, m). | 444 | 3a |
| 6 | 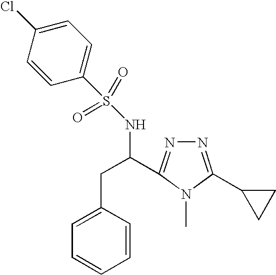 4-chloro-N-[1-(5-cyclopropyl-4- methyl-4H-1,2,4-triazol-3-yl)-2- phenylethyl]benzenesulfonamide | ¹H NMR (300 MHz, CDCl₃) 8.88 (1H, d), 7.50 (4H, m), 7.14 (5H, m), 4.72 (1H, m), 3.46 (3H, s), 3.09 (2H, m), 1.92 (1H, m), 1.03 (2H, m), 0.85 (2H, m). | 416 | 4a |
| 7 | 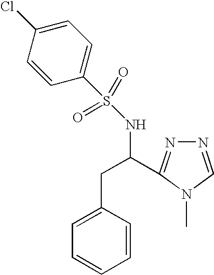 4-chloro-N-[1-(4-methyl-4H- 1,2,4-triazol-3-yl)-2- phenylethyl]benzenesulfonamide | ¹H NMR (300 MHz, CDCl₃) 8.92 (1H, d), 8.42 (1H, s), 7.57 (4H, m), 7.19 (5H, m), 4.77 (1H, m), 3.49 (3H, s), 3.16 (2H, m) | 376 | 5a |
| 8 | 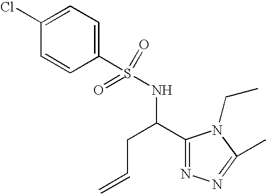 4-chloro-N-[1-(4-ethyl-5- methyl-4H-1,2,4-triazol-3- yl)but-3-en-1-yl]benzene- sulfonamide | ¹H NMR (300 MHz, DMSO) 8.88 (1H, d), 7.80 (2H, d), 7.70 (2H, d), 5.64 (1H, m), 4.99 (2H, m), 4.70 (1H, m), 4.12 (2H, m), 2.66 (1H, m), 2.57 (3H, s), 2.46 (1H, m), 1.30 (3H, t) | 354 | 6a |

Examples 9 and 10 were generated from Example 8 as described below:

### Example 9

### 4-Chloro-N-[1-(4-ethyl-5-methyl-4H-1,2,4-triazol-3-yl)butyl]benzenesulfonamide

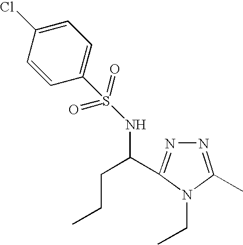

To a solution of 4-chloro-N-[1-(4-ethyl-5-methyl-4H-1,2,4-triazol-3-yl)but-3-en-1-yl]benzenesulfonamide (Example 8, 110 mg) in EtOAc (5 mL) was added Pd/C (5%, 33 mg). The reaction mixture was placed under a hydrogen atmosphere and stirred overnight. The crude mixture was filtered, concentrated and purified using reverse phase HPLC to yield 4-chloro-N-[1-(4-ethyl-5-methyl-4H-1,2,4-triazol-3-yl)butyl]benzenesulfonamide (60 mg) ¹H NMR (300 MHz, MeOD): 7.84 (2H, d), 7.61 (2H, d), 4.68 (1H, t), 4.32 (2H, m), 2.68 (3H, s), 1.85 (1H, m), 1.70 (1H, m), 1.47 (3H, t), 1.30 (1H, m), 1.22 (1H, m), 0.82 3(H, t). M/Z 356.

### Example 10

### 4-Chloro-N-[2-cyclopropyl-1-(4-ethyl-5-methyl-4H-1,2,4-triazol-3-yl)ethyl]benzenesulfonamide

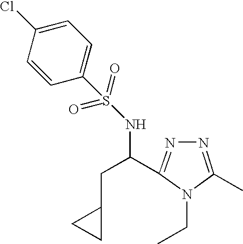

To a 25-mL round bottom flask with a magnetic stir bar and DCM (2 mL) was added diethyl zinc (2 mL, 1.0 M in hexane). The reaction mixture was cooled to 0° C. and TFA (0.091 mL) was added drop wise. The reaction was stirred at this temperature for 20 min followed by addition of CH₂I₂ (0.095 mL). The reaction was stirred for another 20 mins before 4-chloro-N-[1-(4-ethyl-5-methyl-4H-1,2,4-triazol-3-yl)but-3-en-1-yl]benzenesulfonamide (Example 8, 140 mg) was added. The reaction was warmed to room temperature and stirred overnight. The reaction mixture was quenched with a saturated aqueous solution of NH₄Cl and extracted with EtOAc (3×20 mL). The combined organic layers were washed with dried over Na₂SO₄, concentrated and purified using reverse phase HPLC to yield 4-chloro-N-[2-cyclopropyl-1-(4-ethyl-5-methyl-4H-1,2,4-triazol-3-yl)ethyl]benzenesulfonamide. ¹H NMR (300 MHz, MeOD) 7.69 (2H, d), 7.49 (2H, d), 4.54 (1H, t), 3.99 (2H, m), 2.31 (3H, s), 1.85 (1H, m), 1.61 (1H, m), 1.28 (3H, t), 0.49 (1H, m), 0.33 (1H, m), 0.23 (1H, m), 0.02 (1H, m), −0.15 (1H, m). Ma 368.

### Example 11

### 4-Chloro-N-[1-(4-ethyl-5-trifluoromethyl-4H-[1,2,4]triazol-3-yl)-ethyl]-benzenesulfonamide

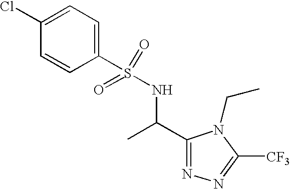

Example 11 was prepared by a two-step procedure described below:
Step I: Generation of [1-(4-ethyl-5-trifluoromethyl-4H-[1,2,4]triazol-3-yl)-ethyl]-carbamic acid tert-butyl ester:

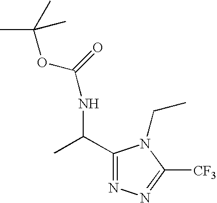

[D]-[1-(5-Trifluoromethyl-[1,3,4]oxadiazol-2-yl)-ethyl]-carbamic acid tert-butyl ester (Intermediate 7a, 0.1 g, 0.35 mmol) and methylammonium trifluoroacetate (0.79 g, 5 mmol) were suspended in a 2 M solution of ethyl amine in methanol (2.5 mL, 5 mmol) in a sealed tube and heated at 150° C. overnight. After cooling to rt, the reaction mixture was partitioned between EtOAc (30 mL) and saturated aq. NaHCO₃ (30 mL). The layers were separated and the aqueous layer extracted with EtOAc (20 mL). The combined organic layers were washed with brine (15 mL), dried (MgSO₄), filtered and evaporated. The residue was purified by silica-gel column chromatography (1-1.5% MeOH/CHCl₃) to afford the product, 30 mg. ¹H NMR (300 MHz, CDCl₃): 5.12-4.99 (m, 2H), 4.31-4.13 (m, 2H), 1.67 (d, J=7.14 Hz, 3H), 1.44-1.40 (m, 12H). (M+1)/Z=309.2.

### Step II

To a solution of [1-(4-ethyl-5-trifluoromethyl-4H-[1,2,4]triazol-3-yl)-ethyl]-carbamic acid tent-butyl ester (26 mg, 0.084 mmol) in anhydrous dioxane (1 mL) added 4M HCl in dioxane (1 mL) under N₂ atmosphere. It was stirred at rt for 3 h. The solvent removed by evaporation and residue dried under vacuum afforded the amine hydrochloride salt. To this was added CH₂Cl₂ (2 mL) under N₂ atmosphere followed by Et₃N (0.025 mL, 0.19 mmol) at 0° C. After stirring for 5 min, the solution became clear and to this was added p-chlorobenzene sulfonyl chloride (0.018 g, 0.084 mmol). The resulting mixture was allowed to stir at rt for 16 h. The reaction mixture was diluted with CH₂Cl₂ (15 mL), quenched with water (5 mL), the organic layer separated and washed with brine (10 mL), dried over MgSO₄, filtered, evaporated and the residue purified by silica-gel chromatography (1% MeOH/CHCl₃) to afford the product, 20 mg.

Examples 12 and 13, shown in Table 2, were prepared in a manner analogous to that described for Example 11 using Intermediates 8a and 9a as indicated in TABLE 2.

TABLE 2

| Ex | Structure | NMR | MS | Intermediate |
|---|---|---|---|---|
| 12 | 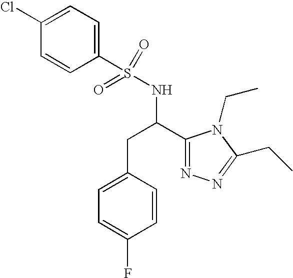 4-chloro-N-[1-(4,5-diethyl-4H- [1,2,4]triazol-3-yl)-2-(4-fluoro- phenyl)-ethyl]-benzenesulfonamide | ¹H NMR (300 MHz, DMSO- d₆): 8.86 (bs, 1H), 7.55-7.46 (m, 4H), 7.13-6.94 (m, 4H), 4.56-4.48 (m, 1H), 3.78-3.60 (m, 2H), 3.20-2.97 (m, 2H), 1.17 (t, J = 7.41 Hz, 3H), 0.89 (t, J = 7.41 Hz, 3H) | 436 | 8a |
| 13 | 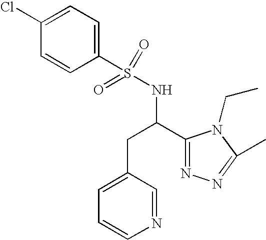 4-chloro-N-[1-(4-ethyl-5-methyl- 4H-1,2,4-triazol-3-yl)-2-pyridin-3- ylethyl]benzenesulfonamide | ¹H NMR (300 MHz, CDCl₃): 8.39-7.81 (m, 5H), 7.43-7.35 (m, 3H), 7.06-7.01 (m, 1H), 4.51 (bs, 1H), 3.87-3.73 (m, 1H), 3.60-3.46 (m, 1H), 3.38-3.30 (m, 1H), 3.1-3.0 (m, 1H), 2.33 (s, 3H), 0.92- 0.81 (m, 3H) | 405 | 9a |

### Example 14

### [D]-4-Chloro-N-{1-[4-ethyl-5-(2,2,2-trifluoro-ethyl)-4H-[1,2,4]triazol-3-yl]-ethyl}-benzenesulfonamide

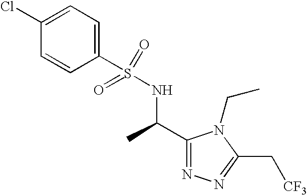

Example 14 was prepared from intermediate 10a in a two-step procedure described below:

### Step I:

### Generation of [D]-1-[4-ethyl-5-(2,2,2-trifluoro-ethyl)-4H-[1,2,4]triazol-3-yl]-ethylamine hydrochloride from Intermediate 10a

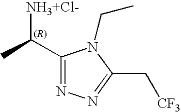

To a solution of [D]-1-[4-ethyl-5-(2,2,2-trifluoro-ethyl)-4H-[1,2,4]triazol-3-yl]-ethyl}-carbamic acid tert-butyl ester (Intermediate 10a, 0.21 g, 0.66 mmol) in anhydrous dioxane (2 mL) was added 4M HCl in dioxane (3.3 mL, 13.2 mmol) under N₂ atmosphere. It was stirred at rt for 4 h. The solvent was removed by evaporation and the residue dried under vacuum to afford the product as a white solid (0.17 g, 98%). ¹H NMR (300 MHz, DMSO-d₆+D₂O): 4.63 (q, J=6.33 Hz, 1H), 4.02-3.92 (m, 4H), 1.54 (d, J=6.87 Hz, 3H), 1.21 (t, J=6.87 Hz, 3H). M/Z=222.2.

### Step II:

To a suspension of [D]-1-[4-ethyl-5-(2,2,2-trifluoro-ethyl)-4H-[1,2,4]triazol-3-yl]-ethylamine hydrochloride generated in Step I (0.15 g, 0.6 mmol) in anhydrous CH₂Cl₂ (5 mL) under a N₂ atmosphere was added Et₃N (0.28 mL, 2 mmol) at 0° C. After stirring for 5 min, the solution became clear and to this was added p-chlorobenzene sulfonyl chloride (0.15 g, 0.72 mmol) in CH₂Cl₂ (5 mL) dropwise over 10 min. The resulting mixture was allowed to stir at rt for 16 h. The reaction mixture was then quenched with water (20 mL), the organic layer separated and the aqueous layer extracted with CH₂Cl₂, washed with water (10 mL), brine (10 mL), dried over MgSO₄, filtered, evaporated and the residue purified by silica-gel chromatography (3% MeOH/CHCl₃) to afford Example 14 as a white solid (0.21 g). ¹H NMR (300 MHz, DMSO-d₆): 8.57 (bs, 1H), 7.79-7.76 (m, 2H) 7.66-7.63 (m, 2H), 4.70 (q, J=6.87 Hz, 1H), 4.08-3.95 (m, 4H), 1.27-1.19 (m, 6H). M/Z=396.

### Intermediate 1a

### 4-chloro-N-[1-(5-methyl-1,3,4-oxadiazol-2-yl)-2-phenylethyl]benzenesulfonamide

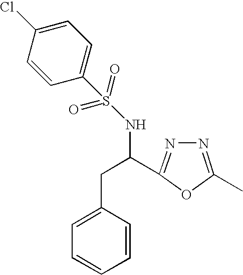

A solution of N-[2-(N-Acetyl-hydrazino)-1-benzyl-2-oxo-ethyl]-4-chloro-benzenesulfonamide (Intermediate 1b, 670 mg) in CH₃CN (10.27 mL) was treated with POCl₃ (1.26 mL, 8.0 eq). The resulting solution was heated to reflux for 4 hours. The reaction mixture was cooled, slowly poured into ice water (100 mL) and extracted with EtOAc (2×30 mL). The combined organic layers were dried over Na₂SO₄ and concentrated to yield crude product as yellow solid which was purified using reverse phase HPLC to afford 4-chloro-N-[1-(5-methyl-1,3,4-oxadiazol-2-yl)-2-phenylethyl]benzenesulfonamide (Intermediate 1a, 250 mg, 40%). Intermediates 2a-6a, described in TABLE 3, were prepared by a method analogous to that described above for Intermediate 1a, using Starting Intermediates 2b-6b, as indicated in TABLE 3.

TABLE 3

| | | | Prepared |
|---|---|---|---|
| | | | from |
| | | | Intermediate |
| Intermediate | Structure | MS | # |
| 2a | 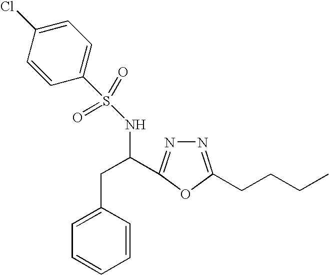 | 419 | 2b |
| | N-[1-(5-Butyl-[1,3,4]oxadiazol-2-yl)-2-phenyl- | | |
| | ethyl]-4-chloro-benzenesulfonamide | | |
| 3a | 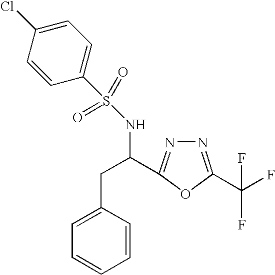 | 431 | 3b |
| | 4-Chloro-N-[2-phenyl-1-(5-trifluoromethyl- | | |
| | [1,3,4]oxadiazol-2-yl)-ethyl]-benzenesulfonamide | | |
| 4a | 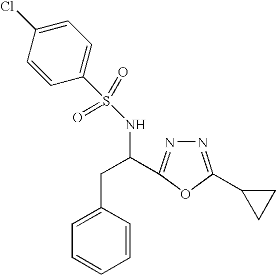 | 403 | 4b |
| | 4-chloro-N-[1-(5-cyclopropyl-1,3,4-oxadiazol-2- | | |
| | yl)-2-phenylethyl]benzenesulfonamide | | |
| 5a | 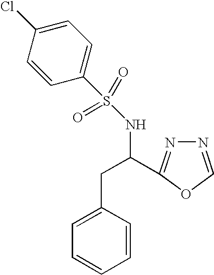 | 363 | 5b |
| | 4-Chloro-N-(1-[1,3,4]oxadiazol-2-yl-2-phenyl- | | |
| | ethyl)-benzenesulfonamide | | |
| 6a | 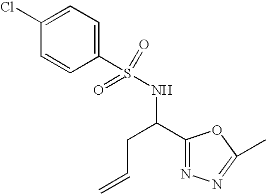 | 327 | 6b |
| | 4-chloro-N-[1-(5-methyl-1,3,4-oxadiazol-2-yl)but- | | |
| | 3-en-1-yl]benzenesulfonamide | | |

### Intermediate 7a

### [D]-[1-(5-Trifluoromethyl-[1,3,4]oxadiazol-2-yl)-ethyl]-carbamic acid tert-butyl ester

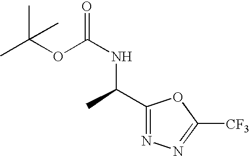

To a suspension of [D]-1-methyl-2-oxo-2-(N-trifluoroacetyl-hydrazino)-ethyl]-carbamic acid tert-butyl ester (Intermediate 7b, 2.13 g, 7.12 mmol) in CH₃CN (70 mL) was added DIEA (7.2 mL, 41.3 mmol) and triphenyl phosphine (7.71 g, 29.4 mmol) followed after 5 min by hexachloroethane (3.9 g, 16.5 mmol). After stirring the mixture at rt for 20 h the solvent was removed by evaporation and the residue partitioned with H₂O (100 mL) and EtOAc (150 mL). The organic phase was separated and the aqueous phase was re-extracted with EtOAc (50 mL). The combined organic phases were washed with H₂O (50 mL), brine (50 mL), dried (MgSO₄) and evaporated, and the residue was purified by silica-gel chromatography (12-20% EtOAc/hexanes) to afford Intermediate 7a (1.19 g).

Intermediates 8a and 9a, shown in TABLE 4, were prepared from 8b′ and 9b′ which is turn were generated from Intermediates 8b and 9b, respectively as described below.

TABLE 4

| Intermediate | | | |
|---|---|---|---|
| a | Structure | NMR | MS |
| 8a | 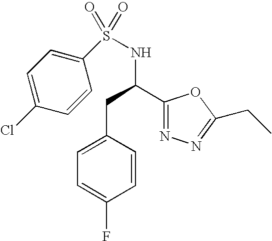 [D]-[1-(5-ethyl-[1,3,4]oxadiazol-2-yl)-2-(4- fluorophenyl)-ethyl]-carbamic acid tert- butyl ester | ¹H NMR (300 MHz, DMSO-d₆): 8.91 (bs, 1H), 7.51 (s, 4H), 7.21- 6.97 (m, 4H), 4.79-4.74 (m, 1H), 3.13-3.00 (m, 2H), 2.66-2.51 (m, 2H), 1.13 (t, J = 7.44 Hz, 3H) | 409 |
| 9a | 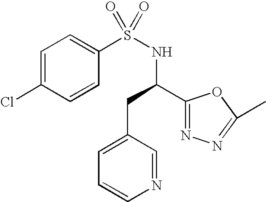 [D]-[1-(5-methyl-[1,3,4]oxadiazol-2-yl)-2- pyridin-3-yl-ethyl]-carbamic acid tert-butyl ester | — | 378 |

### Intermediate 8b′

### [D]-[1-(5-ethyl-[1,3,4]oxadiazol-2-yl)-2-(4-fluoro-phenyl)-ethyl]-carbamic acid tert-butyl ester

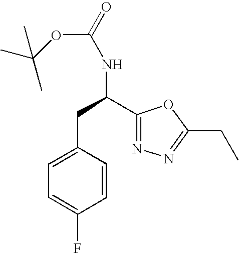

To a solution [D]-[2-(4-fluoro-phenyl)-(1-hydrazinocarbonyl-ethyl)-carbamic acid tert-butyl ester (Intermediate 8b, 4.45 g, 15 mmol) and diisopropylethyl amine (18.2 mL, 105 mmol) in CH₃CN (115 mL) was added propionic anhydride (2.44 mL, 18.75 mmol) under N₂ atm and the mixture was allowed to stir for 2.5 h at room temperature. To this mixture was added PPh₃ (16.2 g, 61.5 mmol), followed by hexachloroethane (8.16 g, 34.5 mmol). After stirring the mixture at rt for 24 h the solvent was removed in vacuo and the residue partitioned with H₂O (200 mL)/EtOAc (300 mL). The organic phase was separated and the aqueous phase was re-extracted with EtOAc (100 mL). The combined organic phases were washed with H₂O (100 mL), brine (100 mL), dried (MgSO₄) and evaporated, and the residue was purified by silica-gel column chromatography (20% ethylacetate in hexanes) gave intermediate 8b′, 4.31 g. ¹H NMR (300 MHz, DMSO-d₆): 7.66-7.07 (m, 5H), 4.95-4.89 (m, 1H), 3.21-3.01 (m, 2H), 2.83 (q, J=7.41 Hz, 2H), 1.30 (s, 9H), 1.22 (t, J=7.69 Hz, 3H). M/Z 335.

### Intermediate 8a

### [D]-4-chloro-N-[1-(5-ethyl-[1,3,4]oxadiazol-2-yl)-2-(4-fluorophenyl)-ethyl]-benzenesulfonamide

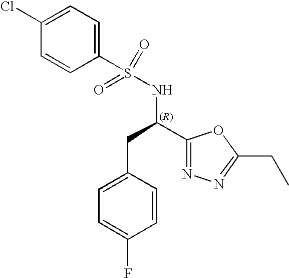

To a solution of [D]-[1-(5-ethyl-[1,3,4]oxadiazol-2-yl)-2-(4-fluoro-phenyl)-ethyl]-carbamic acid tert-butyl ester (intermediate 8b′, 4.18 g, 12.46 mmol) in anhydrous dioxane (20 mL) was added slowly 4M HCl in dioxane (80 mL) under N₂ atm. It was stirred at rt for 4 h. The solvent removed by evaporation and dried under vacuum afforded the product as a gummy oil. To the suspension of this gum (3.27 g, 12 mmol) in dry CH₂Cl₂ (100 mL) under N₂ atm Et₃N (5.6 mL, 40 mmol) was added at 0° C. After stirring for 5 min, p-chloro-benzenesulfonyl chloride (2.66 g, 12.64 mmol) dissolved in dry CH₂Cl₂ (30 mL) was added dropwise to the above mixture for a period of 15 min. The reaction mixture was further allowed to stir at rt for 24 h. It was quenched with H₂O (100 mL), the organic layer separated and the aq. layer extracted with CH₂Cl₂ (2×50 mL). The combined organic phases were washed with H₂O (50 mL), brine (75 mL), dried over MgSO₄ and evaporated, and the residue was purified by silica-gel column chromatography (35-50% ethylacetate in hexanes) gave sulfonamide 2.0 g (40.6%).

### Intermediate 9b′

### tert-butyl [(1R)-1-(5-methyl-1,3,4-oxadiazol-2-yl)-2-pyridin-3-ylethyl]carbamate

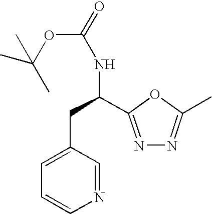

Intermediate 9b′ was generated from 9b following the procedure used for converting 8b to 8b′. M/Z 304.

### Intermediate 9a

### 4-chloro-N-[(1R)-1-(5-methyl-1,3,4-oxadiazol-2-yl)-2-pyridin-3-ylethyl]benzenesulfonamide

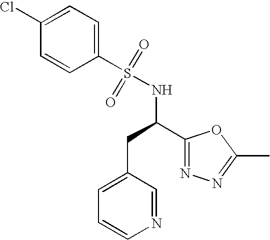

Intermediate 9a was generated from 9b′ by following the method used for generating 8a from 8b′.

### Intermediate 10a

### [D]-1-[4-ethyl-5-(2,2,2-trifluoro-ethyl)-4H-[1,2,4]triazol-3-yl]-ethyl}-carbamic acid tert-butyl ester

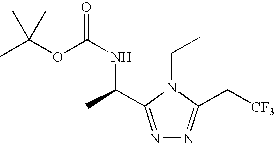

To a solution of [D]-2-tert-Butoxycarbonylamino-N-ethyl-thiopropionimidic acid methyl ester (Starting Material 6, 0.25 g, 1 mmol) and 3,3,3-trifluoropropionic acid hydrazide (Starting Material 7, 0.17 g, 1.2 mmol) in THF (10 mL) was added TFA (0.59 mmol, 0.046 mL) under N₂ atm and heated under reflux for 16 h. The reaction mixture was cooled to rt and then diluted with CH₂Cl₂ (100 mL). The solution was washed with 1% NaOH (100 mL), H₂O (50 mL), brine (20 mL), dried over MgSO₄, filtered and evaporated. The residue was purified by silica-gel chromatography (2% MeOH/CHCl₃) to afford Intermediate 10a (0.25 g, 76%).

### Intermediate 1b

### N-[2-(N′-Acetyl-hydrazino)-1-benzyl-2-oxo-ethyl]-4-chloro-benzenesulfonamide

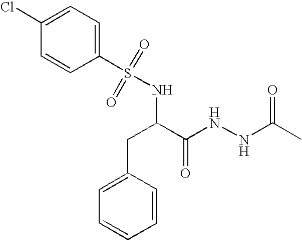

To a solution of N-[(4-chlorophenyl)sulfonyl]phenylalanine (Starting Material 1.3 g, 8.8 mmol) and acetohydrazide (0.85 g, 11.5 mmol) in DMF (14.7 mL) was added Hunig's base (4.6 mL). The resulting solution was stirred for 5 minutes and was treated with HATU (4.37 g, 1.3 mmol). The reaction mixture was stirred for 12 hours at room temperature, quenched with water and extracted with EtOAc (3×200 mL). The combined organic layers were washed with HCl (aq. 1N, 200 mL), dried over Na₂SO₄ and concentrated to yield Intermediate 1b as a yellow solid. The crude product was used directly in the next step. M/Z=395.

### Intermediate 5b

### N-[2-(N′-formyl-hydrazino)-1-benzyl-2-oxo-ethyl]-4-chloro-benzenesulfonamide

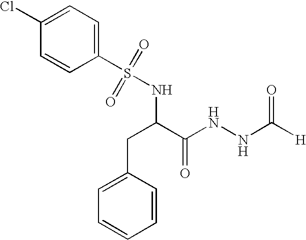

A solution of (Starting Material 1, 500 mg, 1.18 mmol) in formic acid (5 mL) was heated to 130° C. for one hour in a microwave. The reaction mixture was quenched with a saturated aqueous solution of NaHCO₃ and extracted with EtOAc (3×20 mL). The combined organic layers were washed with HCl (aq. 1N, 200 mL), dried over Na₂SO₄ and concentrated to yield Intermediate 5b. The crude product was used directly in the next step. M/Z=381.

### Intermediate 7b

### [D]-1-methyl-2-oxo-2-(N-trifluoroacetyl-hydrazino)-ethyl]-carbamic acid tert-butyl ester

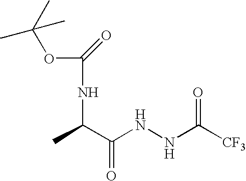

To a solution of [D]-(1-hydrazinocarbonyl-ethyl)-carbamic acid tert-butyl ester (starting material 1, 3 g, 15 mmol) and DIEA (2.85 ml, 16.5 mmol) in CH₃CN (75 mL) was added trifluoroacetic anhydride (2 mL, 15 mmol) at −45° C. under N₂ atmosphere. It was gradually warmed to rt and further stirred for 30 min at rt. The solvent was removed by evaporation and the residue partitioned with H₂O (75 mL) and EtOAc (100 mL). The organic phase was separated and the aqueous phase was re-extracted with EtOAc (50 mL). The combined organic phases were washed with H₂O (25 mL), brine (25 mL), dried (MgSO₄) and evaporated, and the residue was purified by silica gel chromatography (40% EtOAc/hexanes) gave the product 2.87 g (64%). Intermediates 2b-4b, 6b, 8b and 9b, shown in Table 5, were prepared in a manner analogous to that used to prepare Intermediate 7b, described below, using the starting material indicated in TABLE 5 and the appropriate commercially available anhydride or acid chloride.

TABLE 5

| | | | Starting |
|---|---|---|---|
| Intermediate | Structure | M/Z | Material |
| 2b | 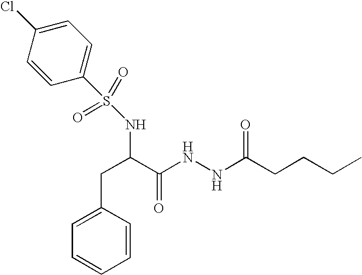 | 437 | 1 |
| | N-[1-Benzyl-2-oxo-2-(N′-pentanoyl-hydrazino)- | | |
| | ethyl]-4-chloro-benzenesulfonamide | | |
| 3b | 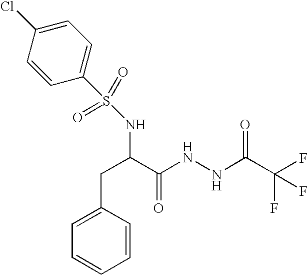 | 449 | 1 |
| | N-{1-Benzyl-2-oxo-2-[N′-(2,2,2-trifluoro- | | |
| | acetyl)-hydrazino]-ethyl}-4-chloro- | | |
| | benzenesulfonamide | | |
| 4b | 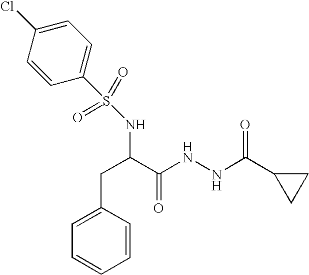 | 421 | 1 |
| | N-[1-Benzyl-2-(N′-cyclopropanecarbonyl- | | |
| | hydrazino)-2-oxo-ethyl]-4-chloro- | | |
| | benzenesulfonamide | | |
| 6b | 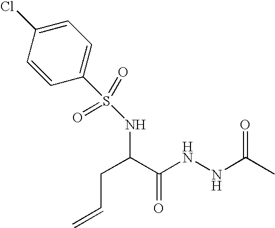 | 345 | 2 |
| | N-[1-(N′-Acetyl-hydrazinocarbonyl)-but-3- | | |
| | enyl]-4-chloro-benzenesulfonamide | | |
| 8b | 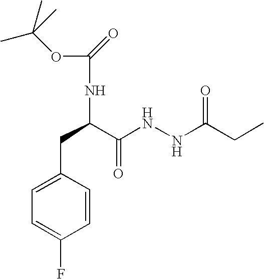 | 353 | 4 |
| | [(R)-1-(4-Fluoro-benzyl)-2-oxo-2-(N′-propionyl- | | |
| | hydrazino)-ethyl]-carbamic acid tert-butyl ester | | |
| 9b | 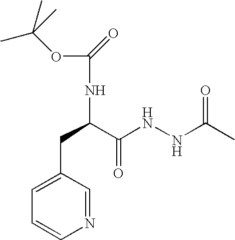 | 322 | 5 |
| | [(R)-2-(N′-Acetyl-hydrazino)-2-oxo-1-pyridin-3- | | |
| | ylmethyl-ethyl]-carbamic acid tert-butyl ester | | |

### Preparation of Starting Materials

### Starting Material 1

### 4-Chloro-N-(−1-hydrazinocarbonyl-2-phenyl-ethyl)-benzenesulfonamide

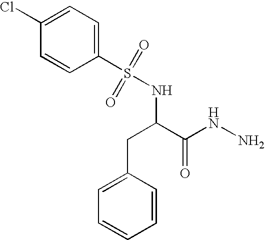

The title compound was prepared in 3 steps starting with commercially available phenylalanine. M/Z 353.

### Step I: Preparation of Starting Material 1a

### N-[(4-chlorophenyl)sulfonyl]phenylalanine

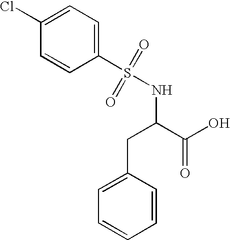

To a suspension of D-phenylalanine (100 g, 606 mmol) in a solution of NaHCO₃ (103 g, 1.2 mol) in H₂O was added p-chlorophenylsulfonyl chloride (140.6 g, 666 mmol). After stirring overnight at room temperature, the reaction mixture was filtered and washed with methylene chloride. The resulting mixture was acidified with HCl and extracted with EtOAc, dried, filtered, and concentrated to give the title compound (109 g). M/Z=339.

### Step 11: Preparation of Starting material 1b

### Methyl N-[(4-chlorophenyl)sulfonyl]phenylalaninate (Starting material 1b)

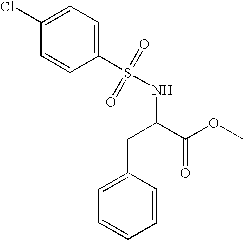

To a suspension of phenylalanine 10 g (60.6 mmol) in a solution of MeOH (120 mL) was treated with thionyl chloride (14.42 g, 121 mmol). After stirring overnight at room temperature, the reaction mixture was concentrated and diluted with EtOAc (300 mL). The organic layer was washed with a saturated aqueous solution of NaHCO₃, dried over Na₂SO₄ and concentrated to yield a yellow solid 10 g. M/Z=353.

### Step III: Generation of Starting material 1: 4-Chloro-N-(−1-hydrazinocarbonyl-2-phenylethyl)-benzenesulfonamide

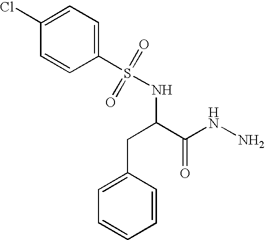

Starting Material 1b was converted to Starting Material 1 by following the procedure for generation of starting material 3 described below. M/Z 353.

### Starting Material 2

### 4-Chloro-N-(1-hydrazinocarbonyl-but-3-enyl)-benzenesulfonamide

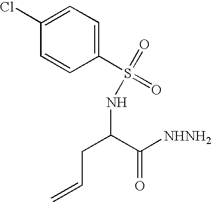

Starting material 2 was generated analogously to the described for Starting Material 1 using the appropriate commercially available starting materials. M/Z=303

### Starting Material 3

### [D]-(1-Hydrazinocarbonyl-ethyl)-carbamic acid tent-butyl ester

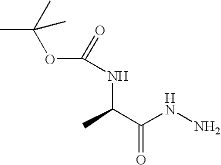

To a solution of Boc-D-alanine methyl ester (10 g, 49 mmol) in ethanol (250 mL) was added NH₂NH₂.H₂O (35.6 mL, 735 mmol) and stirred at rt for 16 h. The solvent was removed by evaporation and the residue triturated with hexane to yield a white foamy solid, which was filtered and dried under vacuum. Yield: 9.8 g. ¹H NMR (300 MHz, DMSO-d₆): 8.97 (s, 1H), 6.86-6.84 (m, 1H), 4.18 (s, 2H), 3.96-3.86 (m, 1H), 1.36 (s, 9H), 1.13 (d, J=7.14 Hz, 3H). M/Z=203

### Starting Material 4

### [D]-[2-(4-Fluoro-phenyl)-(1-hydrazinocarbonyl-ethyl)-carbamic acid tert-butyl ester

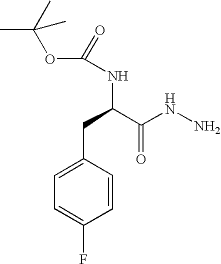

To a solution of Boc-4-fluoro-D-phenyl alanine (5.0 g, 17.65 mmol) in MeOH:toluene (50 mL:50 mL) was added dropwise TMSCHN₂ (17.65 mL, 35.31 mmol) at 0° C. The resulting mixture was stirred at 0° C. for 2 h. The solvent was removed by evaporation and the product dried under vacuum to yield the corresponding ester as a gummy oil (Starting Material 4a). A solution of Boc-4-fluoro-D-phenyl alanine methylester (5.19 g, 17.5 mmol) in ethanol (150 mL) was treated with hydrazine hydrate (12.7 mL, 262 mmol). After stirring for 16 h at rt, the solvent removed by evaporation and the product dried under vacuum to yield Starting Material 4 (5.0 g.

¹H NMR (300 MHz, DMSO-d₆): 9.12 (bs, 1H), 7.29-6.93 (m, 5H), 4.22 (bs, 2H), 4.08-4.02 (m, 1H), 2.87-2.67 (m, 2H), 1.28 (s, 9H). M/Z=297

### Starting Material 5

### [D]-(1-Hydrazinocarbonyl-2-pyridin-3-yl-ethyl)-carbamic acid tert-butyl ester

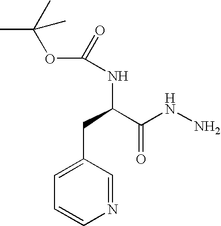

[D]-2-tent-Butoxycarbonylamino-3-pyridin-3-yl-propionic acid (2.12 g, 8 mmol) was dissolved in CH₃CN (20 mL) and DMF (8 mL). HOBt (1.29 g, 9.6 mmol) was added in one portion followed by EDCI (1.84 g, 9.6 mmol). The mixture was stirred at rt for 2 h, then slowly added to a solution of hydrazine monohydrate (0.77 ml, 16 mmol) in CH₃CN (8 mL) while the temperature was maintained at 0-10° C. The reaction mixture was further stirred at rt for 24 h. It was quenched with water (50 mL) and extracted with EtOAc (2×100 mL). The organic layers were washed with aq. NaHCO₃, water (25 mL), brine (25 mL), dried over MgSO₄, filtered and evaporated to dryness to afford the product, 1.31 g. ¹H NMR (300 MHz, DMSO-d₆): 9.16 (bs, 1H), 8.44-8.39 (m, 2H), 7.65-7.63 (m, 1H), 7.31-7.27 (m, 1H), 7.04-7.01 (m, 1H), 4.25 (bs, 2H), 4.14-4.06 (m, 1H), 2.92-2.70 (m, 2H), 1.27 (s, 9H). M/Z=280.2.

### Starting Material 6

### D]-2-test-Butoxycarbonylamino-N-ethyl-thiopropionimidic acid methyl ester

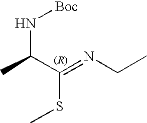

Starting Material 6 was generated in 3 steps from commercially available Boc-D-alanine N-hydroxysuccinimide ester as described below.

### Step I

### Generation of [D]-(1-ethylcarbamoyl-ethyl)-carbamic acid tent-butyl ester (Starting Material 6a)

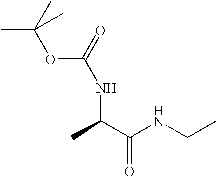

To Boc-D-alanine N-hydroxysuccinimide ester (4.95 g, 17.3 mmol) was added 70% aq. ethyl amine (60 mL) via a dropping funnel over 15 min at 0° C. The mixture was then allowed to stir at room temperature for 24 h. The solvent was removed by evaporation and the residue purified by silica-gel chromatography (2% MeOH/CHCl₃) to afford 3.0 g. ¹H NMR (300 MHz, DMSO-d₆): 7.72 (bs, 1H), 6.78 (d, J=7.71 Hz, 1H), 3.91-3.86 (m, 1H), 3.10-2.98 (m, 2H), 1.37 (s, 9H), 1.14 (d, J=7.14 Hz, 3H), 0.99 (t, J=7.14 Hz, 3H). M/Z 216.2.

### Step II: Generation of (R)-2-tert-Butoxycarbonylamino-N-ethyl-thiopropionimidic acid (Starting Material 6b)

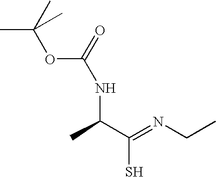

To a solution of [D]-(1-ethylcarbamoyl-ethyl)-carbamic acid tert-butyl ester (2.95 g, 10 mmol) in dry toluene (70 mL) under a N₂ atmosphere was added Lawesson's reagent (2.42 g, 6 mmol) and the resulting mixture was refluxed for 2 h. The reaction mixture was cooled to room temperature, EtOAc (300 mL) and 10% aq. KOH (200 mL) were added and stirred for 30 min. The organic layer was separated, washed with water (100 mL), brine (100 mL), dried over MgSO₄, filtered, concentrated and the residue purified by silica-gel chromatography (15% EtOAc/hexanes) to afford the product 2.16 g. ¹H NMR (300 MHz, DMSO-d₆): 9.85 (bs, 1H), 6.83 (bs, 1H), 4.33-4.28 (m, 1H), 3.53-3.48 (m, 2H), 1.37 (s, 9H), 1.22 (d, J=6.87 Hz, 3H), 1.11 (t, J=7.27 Hz, 3H). (M+1)/Z=233.1, 133.1.

### Step III: (R)-2-tert-Butoxycarbonylamino-N-ethyl-thiopropionimidic acid methyl ester (Starting Material 6)

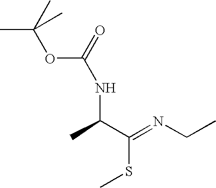

To a solution of [D]-(1-ethylthiocarbamoyl-ethyl)-carbamic acid tert-butyl ester (1.69 g, 7.27 mmol) in anhydrous DMF (10 mL) was added Na₂CO₃ (0.84 g, 8 mmol) under a N₂ atmosphere. After stirring for 5 min, methyl iodide (0.5 mL, 8 mmol) was added and the resulting mixture was stirred at rt for 16 h. The solvent was removed by evaporation and the residue partitioned between EtOAc (200 mL) and H₂O (100 mL). The organic layer was separated, washed with water (3×100 mL), brine (50 mL), dried over MgSO₄, filtered and evaporated to dryness. The residue was purified by silica-gel chromatography (10% EtOAc/hexanes) to afford the product 0.94 g. ¹H NMR (300 MHz, CDCl₃): 5.80 (bs, 1H), 4.61-4.52 (m, 1H), 3.48-3.26 (m, 2H), 2.45 (s, 3H), 1.44 (s, 9H), 1.34 (d, J=6.87 Hz, 3H), 1.23 (t, J=7.29 Hz, 3H). M/Z 246.

### Starting Material 7

### 3,3,3-trifluoropropanohydrazide

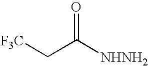

Starting Material 7 was prepared from commercially available 3,3,3-trifluoropropionic acid in two steps as described below.

### Step I

To a solution of 3,3,3-trifluoro propionic acid (4.0 g, 31.87 mmol) in MeOH:toluene (20 mL:20 mL) was added trimethylsilyl diazomethane (2.0M solution in ether, 32 mL, 63.74 mmol) dropwise at 0° C. via a dropping funnel over 10 min under a N₂ atmosphere. The resulting mixture was stirred at 0° C. for 2 h.

### Step II

To the above mixture was added anhydrous NH₂NH₂ (10 mL, 320 mmol) slowly at 0° C. and then allowed to stir at rt overnight. The solvent was removed by evaporation and the residue purified by silica-gel chromatography (4-5% MeOH/CHCl₃) to afford the product 1.99 g. ¹H NMR (300 MHz, DMSO-d₆): 9.34 (bs, 1H), 4.37 (bs, 2H), 3.17 (q, J=11.34 Hz, 2H). M/Z 142.

### Example 15

### 4-chloro-N-[1-(4-ethyl-5-methoxy-4H-1,2,4-triazol-3-yl)-2-pyridin-2-ylethyl]benzenesulfonamide

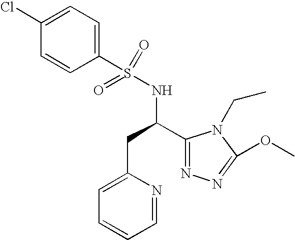

To a solution of [D]-4-chloro-N-[1-(4-ethyl-5-methanesulfonyl-4H-[1,2,4]triazol-3-yl)-2-pyridin-2-yl-ethyl]-benzenesulfonamide (Intermediate 11a, 0.33 g, 0.71 mmol) in dry THF (5 mL) was added sodium methoxide (0.77 g, 14.2 mmol) under N₂ atm and stirred at rt for 4 days. The reaction mixture was quenched with aq. NH₄Cl (20 mL) and extracted with EtOAc (2×50 mL). The combined organic layers were dried over MgSO₄, filtered and concentrated to yield the crude product, which was purified by silica-gel chromatography (2.5-3% methanol in chloroform) to afford Example 15 (0.12 g). ¹H NMR (300 MHz, DMSO-d₆): 8.82 (bs, 1H), 8.37-8.36 (m, 1H), 7.61-7.51 (m, 5H), 7.16-7.12 (m, 2H), 5.00-4.95 (m, 1H), 3.92 (s, 3H), 3.70-3.57 (m, 2H), 3.40-2.99 (m, 2H), 1.06 (t, J=7.54 Hz, 3H). M/Z=421

Examples 16 and 17 in TABLE 6 were made in an analogous manner to that described for Example 15 using the appropriate intermediates

TABLE 6

| Example | Structure | NMR | M/Z |
|---|---|---|---|
| 16 | 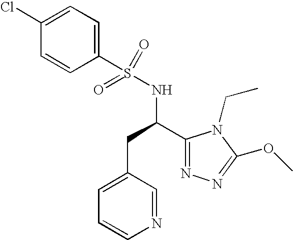 4-Chloro-N-[(R)-1-(4-ethyl-5-methoxy- 4H-[1,2,4]triazol-3-yl)-2-pyridin-3-yl- ethyl]benzenesulfonamide | ¹H NMR (300 MHz, DMSO-d₆): 8.83-8.80 (m, 1H), 8.36-8.33 (m, 2H), 7.57-7.49 (m, 5H), 7.22- 7.17 (m, 1H), 4.59-4.52 (m, 1H), 3.94 (s, 3H), 3.57 (q, J = 7.14 Hz, 2H), 3.21-297 (m, 2H), 0.93 (t, J = 7.17 Hz, 3H) | 421 |
| 17 | 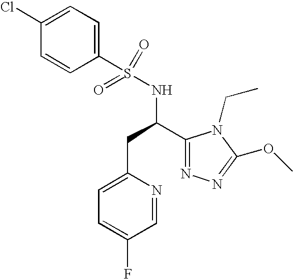 [D]-4-chloro-N-[1-(4-ethyl-5-methoxy- 4H-1,2,4-triazol-3-yl)-2-(5-fluoro- pyridin-2-yl)ethyl]benzenesulfonamide | ¹H NMR (300 MHz, MeOD) 8.14 (1H, d), 7.52 (2H, d), 7.36 (1H, d), 7.30 (1H, m), 7.10 (1H, dd), 4.94 (1H, t), 3.94 (3H, s), 3.73 (2H, m), 3.26 (1H, m), 3.08 (1H, dd), 1.10 (3H, t) | 439 |

### Preparation of Intermediates

### Intermediate 11a

### [D]-4-chloro-N-[1-(4-ethyl-5-methanesulfonyl-4H-[1,2,4]triazol-3-yl)-2-pyridin-2-yl-ethyl]-benzenesulfonamide

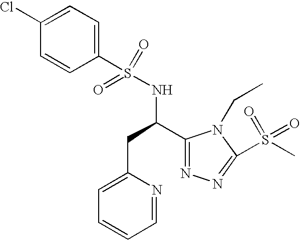

A solution of Potassium permanganate (KMNO₄, 0.168 g, 1.06 mmol) in H₂O (8 mL) was added to a solution of [D]-4-chloro-N-[1-(4-ethyl-5-methylsulfanyl-4H-[1,2,4]triazol-3-yl)-2-pyridin-2-yl-ethyl]-benzenesulfonamide (Intermediate 11b, 0.311 g, 0.71 mmol) in AcOH (4 mL) dropwise at rt over 5 min. After stirring for 3 h, sodium hydrogen sulfite was added until the purple colour discharged. The reaction mixture was extracted with chloroform (2×75 mL) and then the organic layer was washed with saturated aq. NaHCO₃ (50 mL), dried over MgSO₄, filtered and concentrated to afford Intermediate 11a (0.33 g). ¹H NMR (300 MHz, CDCl₃): 9.12 (bs, 1H), 8.35-8.33 (m, 1H), 7.61-7.54 (m, 5H), 7.17-7.15 (m, 2H), 5.19-5.14 (m, 1H), 4.27-4.19 (m, 2H), 3.54 (s, 3H), 3.46-3.10 (m, 2H), 1.29 (t, J=7.14 Hz, 3H). M/Z=469.

Intermediates 12a and 13a in TABLE 7 were made in an analogous manner to that described for Intermediate 11a using the appropriate intermediates.

TABLE 7

| Intermediate | | | |
|---|---|---|---|
| a | Structure | NMR | MS |
| 12a | 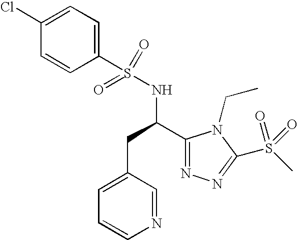 [D]-4-chloro-N-{1-[4-ethyl-5- (methylsulfonyl)-4H-1,2,4-triazol-3- yl]-2-pyridin-3- ylethyl}benzenesulfonamide | ¹H NMR (300 MHz, CDCl₃): 8.47-8.46 (m, 1H), 8.33 (bs, 1H), 7.60-7.57 (m, 2H), 7.41- 7.10 (m, 5H), 4.72-4.67 (m, 1H), 4.13-4.05 (m, 2H), 3.46 (s, 3H), 3.38-3.18 (m, 2H), 1.10 (t, J = 7.41 Hz, 3H) | 469 |
| 13a | 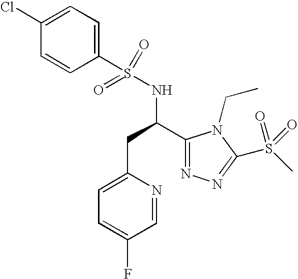 [D]-4-chloro-N-[1-[4-ethyl-5- (methylsulfonyl)-4H-1,2,4-triazol-3- yl]-2-(5-fluoropyridin-2- yl)ethyl]benzenesulfonamide | — | 487 |

### Intermediate 11b

### [D]-4-Chloro-N-[1-(4-ethyl-5-methylsulfanyl-4H-[1,2,4]triazol-3-yl)-2-pyridin-2-yl-ethyl]-benzenesulfonamide

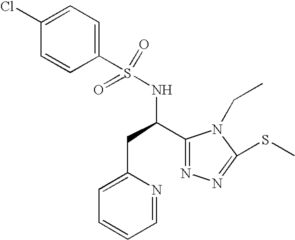

To [D]-[1-(4-Ethyl-5-methylsulfanyl-4H-[1,2,4]triazol-3-yl)-2-pyridin-2-yl-ethylamine dihydrochloride (Intermediate 11c, 3.35 g, 9.98 mmol) was added CH₂Cl₂ (2.5 mL) and pyridine (62 mL). After cooling to 0° C., a solution of 4-chlorobenzenesulfonyl chloride (2.1 g, 9.98 mmol) in CH₂Cl₂ (10 mL) was added dropwise. The reaction mixture was stirred at 24 h. The solvent was removed by evaporation, diluted with CH₂Cl₂ (200 mL) and washed with water (3×25 mL) and brine (50 mL). The organic layer was dried over Na₂SO₄ and concentrated in vacuo to give the residue, which was purified by flash column chromatography using silica-gel (2.5-3.5% MeOH/CHCl₃) to give the product 1.29 g. ¹H NMR (300 MHz, DMSO-d₆): 8.93 (bs, 1H), 8.37-8.36 (m, 1H), 7.63-7.49 (m, 5H), 7.16-7.12 (m, 2H), 5.10-5.05 (m, 1H), 3.87-3.67 (m, 2H), 3.43-3.06 (m, 2H), 2.54 (s, 3H), 1.09 (t, J=7.14 Hz, 3H). MS (or M/Z)=437.

Intermediates 12b and 13b in TABLE 8 were made in an analogous manner to that described for Intermediate 11b.

TABLE 8

| INT b | Structure | NMR | MS |
|---|---|---|---|
| 12b | 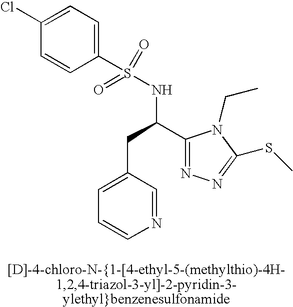 | ¹H NMR 300 MHz, CDCl₃) 8.40-8.33(m, 2H), 7.66- 6.90(m, 7H), 4.62-4.59(m, 1H), 3.82-3.20(m, 4H), 2.65 (s, 3H), 0.92(t, J = 7.4 Hz, 3H) | 437 |
| 13b | 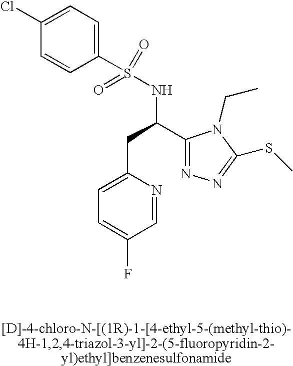 | ¹H NMR (300 MHz, MeOD) 8.27(1H, d), 7.64 (2H, d), 7.46(1H, d), 7.40 (1H, dd), 7.23(1H, dd), 5.15 (1H, t), 4.01(1H, m), 3.90 (1H, m), 3.40(1H, m), 3.30 (1H, m), 2.63(3H, s), 1.23 (3H, t) | 455 |

### Intermediate 11c

### (1R)-1-[4-ethyl-5-(methylthio)-4H-1,2,4-triazol-3-yl]-2-pyridin-2-ylethanamine dihydrochloride

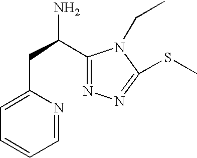

[D]-[1-(4-Ethyl-5-methylsulfanyl-4H-[1,2,4]triazol-3-yl)-2-pyridin-2-yl-ethyl]-carbamic acid tert-butyl ester (Intermediate 11d, 3.66 g, 10 mmol) was cooled in an ice-water bath and 4M HCl solution in dioxane (70 mL, 280 mmol) was added slowly via a dropping funnel. After stirring at rt for 4 h, the reaction mixture was concentrated and filtered through a funnel. The solid material was washed with diethyl ether and dried in vacuo to give the title compound as a yellow powder (3.35 g. ¹H NMR (300 MHz, DMSO-d₆): 9.16 (bs, 2H), 8.76-8.74 (m, 1H), 8.32-8.28 (m, 1H), 7.84-7.74 (m, 2H), 4.18-3.99 (m, 2H), 3.85-3.68 (m, 2H), 2.65 (s, 3H), 1.21 (t, J=7.41 Hz, 3H). M/Z 263

Intermediates 12c and 13c in TABLE 9 were made in an analogous manner to that described for intermediate 11c.

TABLE 9

| Intermediate c | Structure | NMR | MS |
|---|---|---|---|
| 12c | 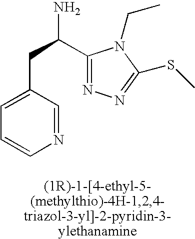 | ¹H NMR (300 MHz, MeOH-d₃) 8.97- 8.11(m, 4H), 5.40(br s, 1H), 4.96(br s, 3H), 4.30-4.00(m, 2H), 3.82-3.60 (m, 2H), 2.82(s, 3H), 1.28(t, J = 7.4 Hz, 3H) | 263 |
| 13c | 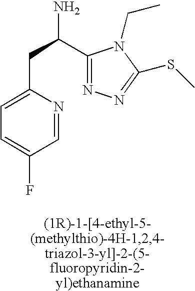 | — | 281 |

### Intermediate 11d

### tert-butyl {(1R)-1-[4-ethyl-5-(methylthio)-4H-1,2,4-triazol-3-yl]-2-pyridin-2-ylethyl}carbamate

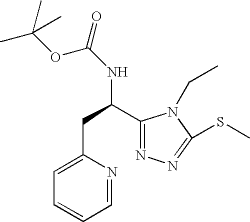

[D]-[1-(4-Ethyl-5-mercapto-4H-[1,2,4]triazol-3-yl)-2-pyridin-2-yl-ethyl-carbamic acid tert-butyl ester (Intermediate 11e, 3.9 g, 11.16 mmol) was dissolved in EtOH (16 mL) and cooled to 0° C. A 1M NaOH solution (47 mL) was added and the reaction mixture was stirred for 10 min before adding MeI (0.76 mL, 12.3 mmol) as a solution in EtOH (4 mL). The reaction mixture was stirred overnight at rt. EtOH was evaporated in vacuo and the aqueous residue was extracted with EtOAc (3×150 mL). The organic layer was dried over MgSO₄, filtered and evaporated to afford the desired product (3.72 g). ¹H NMR (300 MHz, DMSO-d₆): 8.45 (bs, 1H), 7.67-7.63 (m, 2H), 7.27-7.19 (m, 2H), 5.28-5.22 (m, 1H), 3.93-3.89 (m, 2H), 3.56-3.25 (m, 2H), 2.60 (s, 3H), 1.17 (t, J=7.14 Hz, 3H). M/Z=363.

Intermediates 12d and 13d in TABLE 10 were made in an analogous manner to that described for Intermediate 11d using appropriate intermediates.

TABLE 10

| Intermediate d | Structure | NMR | MS |
|---|---|---|---|
| 12d | 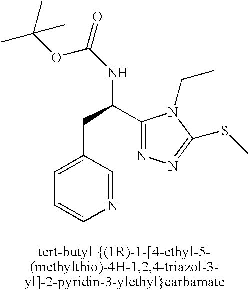 | ¹H NMR (300 MHz, CDCl₃) 8.46 (d, J = 2.5 Hz, 2H), 7.54-7.52(m, 1H), 7.26-7.25(m, 1H), 5.20-5.02 (m, 2H), 3.81-3.29(m, 4H), 2.70 (s, 3H), 1.36(s, 9H), 1.07(t, J = 7.4 Hz, 3H) | 363 |
| 13d | 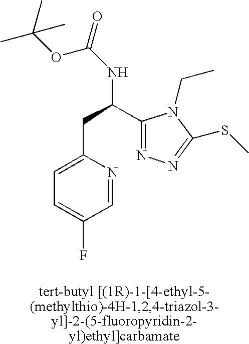 | | 381 |

### Intermediate 11e

The thio semicarbazide (Intermediate 11f, 4.32 g, 11.77 mmol) and 6N NH₃ in MeOH (42 mL) were taken in a sealed tube and placed in an oil bath pre-heated to 65° C. for 7 h. The reaction mixture was cooled to rt and evaporated followed by drying under vacuum yielded the desired product 4.0 g, which was carried on to the next step without further purification. ¹H NMR (300 MHz, DMSO-d₆): 8.47-8.45 (m, 1H), 7.71-7.69 (m, 2H), 7.29-7.21 (m, 2H), 5.28-5.21 (m, 1H), 3.99-3.91 (m, 2H), 3.41-3.16 (m, 2H), 1.30 (s, 9H), 1.18 (t, J=6.6 Hz, 3H).

Intermediates 12e and 13e in TABLE 11 were made in an analogous manner to that described for Intermediate 11e using Intermediates 12d and 13d, respectively.

TABLE 11

| Intermediate | Structure | NMR | MS |
|---|---|---|---|
| 12e | 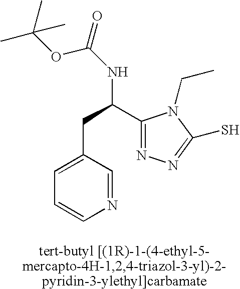 | ¹H NMR (300 MHz, DMSO-d₆): 8.84-8.80(m, 2H), 8.47-8.45(m, 1H), 8.01-7.97(m, 1H), 7.73-7.70 (m, 1H), 5.12-5.05(m, 1H), 3.96- 3.82(m, 2H), 3.48-3.20(m, 2H), 1.22-1.15(m, 12) | 349 |
| 13e | 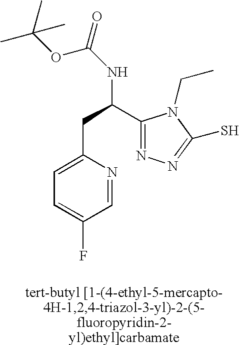 | — | 367 |

### Intermediate 11f

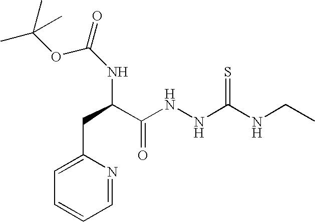

To a solution of [D]-(1-Hydrazinocarbonyl-2-pyridin-2-yl-ethyl)-carbamic acid test-butyl ester (Intermediate 11 g, 4.97 g, 17.73 mmol) in dry CH₃CN (85 mL) was added dropwise ethyl isothiocyanate (1.54 mL, 17.73 mmol) in dry CH₃CN (50 mL) under N₂ atmosphere at rt. It was stirred at rt for 50 h. The solvent was removed by evaporation and the residue purified by silica-gel chromatography (80% EtOAc/hexanes to EtOAc) to afford the product 4.36 g.

### Intermediate 12f

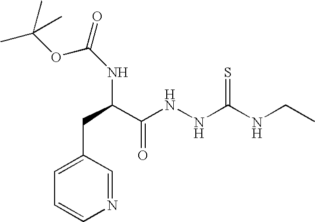

To a solution of [D]-2-tent-Butoxycarbonylamino-3-pyridin-3-yl-propionic acid (commercially available, 12 g, 5 g, 18.77 mmol) and N-ethylhydrazinecarbothioamide (2.68 g, 22.52 mmol) in dry DMF (75 mL) was added DMA (9.8 mL, 56.4 mL) under N₂ atm. The resulting solution was stirred for 5 min and was treated with HATU (8.5 g, 22.52 mmol). The reaction mixture was stirred for 36 h, quenched with water (100 mL) and extracted with EtOAc (2×200 mL). The organic phase was washed with brine (100 mL), dried over MgSO₄, and concentrated. The residue was treated with EtOAc, the precipitated product was filtered and dried under vacuum yielding the title compound (12f, 3 g). ¹H NMR (300 MHz, DMSO-d₆): 10.0 (bs, 1H), 9.34 (bs, 1H), 8.45-8.41 (m, 2H), 7.67-7.64 (m, 1H), 7.52 (bs, 1H), 7.33-7.29 (m, 2H), 4.16-4.09 (m, 1H), 3.54-3.37 (m, 2H), 3.03-2.72 (m, 2H), 1.32 (s, 9H), 1.08 (t, J=7.14 Hz, 3H). M/Z 367.

### Intermediate 13f

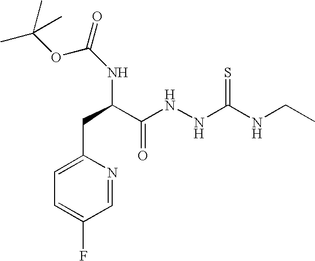

Intermediate 13f was prepared in a manner analogous to that described for Intermediate 11f. M/Z=385.

### Intermediate 11g

### [D]-(1-Hydrazinocarbonyl-2-pyridin-2-yl-ethyl)-carbamic acid tert-butyl ester

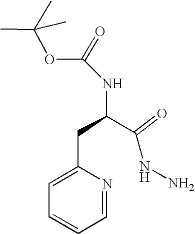

To a solution of Boc-D-2-pyridyl alanine (5.0 g, 18.77 mmol) in MeOH:toluene (50 mL:50 mL) was added dropwise TMSCHN₂ (19 mL, 37.55 mmol) at 0° C. The resulting mixture was stirred at 0° C. for 2 h. The solvent was removed by evaporation and the product dried under vacuum to yield the methyl ester (5.26 g). A solution of the Boc-D-2-pyridyl alanine methylester thus obtained (5.2 g, 18.55 mmol) in ethanol (100 mL) was treated with hydrazine hydrate (13.5 mL, 279 mmol). After stirring for 16 h at rt, the solvent was removed by evaporation and the product dried under vacuum. Yield: 5.0 g. ¹H NMR (300 MHz, DMSO-d₆): 9.09 (bs, 1H), 8.48-8.46 (m, 1H), 7.68-7.65 (m, 1H), 7.26-7.18 (m, 2H), 6.93-6.91 (m, 1H), 4.45-4.29 (m, 1H), 4.20 (bs, 2H), 3.03-2.87 (m, 2H), 1.29 (s, 9H). M/Z 280.

Intermediates 12g and 13g described in TABLE 12 were made in a manner analogous to 11g using Intermediates 12f and 13f, respectively.

TABLE 12

| Intermediate g | Structure | NMR | MS |
|---|---|---|---|
| 12g | 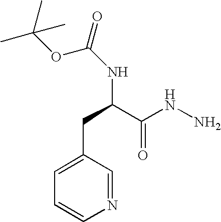 | — | — |
| 13g | 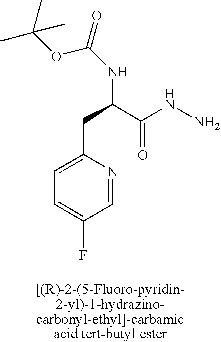 | — | 298 |

### Starting Material 8

N-(tert-butoxycarbonyl)-3-fluoro-D-alaninate was prepared from commercially available N-(tert-butoxycarbonyl)-3-iodo-D-alaninate which was methylated as described above in Step I for Starting Material 7.

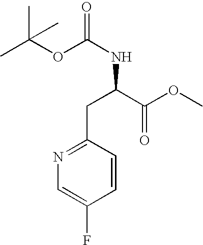

To Zinc dust (0.86 g, 13.4 mmol) was added a solution of methyl N-(tert-butoxycarbonyl)-3-iodo-D-alaninate (2 g, 6.07 mmol) in DMF (4.4 ml). After stirring for 2 h at room temperature, 2-bromo-5-fluoropyridine (1.4 g, 7.9 mmol) and bis(triphenyl phosphine)palladium (II) chloride (213 mg, 0.6 mmol) were added to the resulting solution. The mixture was stirred overnight at room temperature. The reaction was quenched with a saturated ammonia chloride solution and extracted with EtOAc. The combined organic layers were washed, dried over Na₂SO₄ and concentrated. The crude material was purified using silica chromatography (20% to 40% EtOAc/Hexanes) to afford methyl N-(tert-butoxycarbonyl)-3-(6-fluoropyridin-3-yl)-D-alaninate (970 mg). M/Z 298.

### A. Synthesis of Mercapto Reference Compounds and Intermediates: Method A.1

### A.1 Preparation of (R)-4-Chloro-N-[1-(4-ethyl-5-mercapto-4H-1,2,4-triazol-3-yl)-2-phenylethyl]benzenesulfonamide

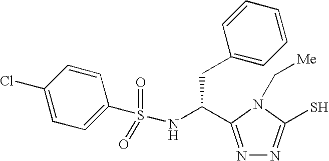

A solution of (R)-2-(2-{[(4-chlorophenyl)sulfonyl]amino}-3-phenylpropanoyl)-N-ethylhydrazinecarbothioamide in NH₃/MeOH (30 mL, 2N) and KOH (30 mL, 10% by weight in water) was heated at reflux for 4 hours. The reaction mixture was cooled in an ice bath and acidified by adding concentrated HCl dropwise to a pH of approximately 3. The resulting white precipitate was filtered and washed with water and air-dried to give 6.5 g (79% yield) of (R)-4-chloro-N-[1-(4-ethyl-5-mercapto-4H-1,2,4-triazol-3-yl)-2-phenylethyl]benzenesulfonamide. M/Z 422.

(R)-2-(2-{[(4-chlorophenyl)sulfonyl]amino}-3-phenylpropanoyl)-N-ethylhydrazinecarbothioamide was prepared as indicated below.

### a. (R)-2-(2-{[(4-Chlorophenyl)sulfonyl]amino}-3-phenylpropanoyl)-N-ethylhydrazinecarbothioamide

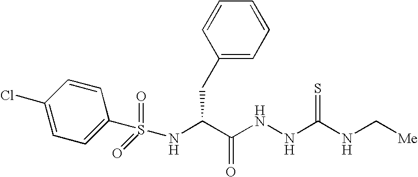

To a solution of (R)—N-[(4-chlorophenyl)sulfonyl]phenylalanine (10 g, 29.5 mmol) and N-ethylhydrazinecarbothioamide (0.4.2 g, 35.3 mmol) in DMF (59 mL) was added N,N diisopropyethylamine (Hunig's Base) (15.9 mL). The resulting solution was stirred for 5 minutes and then was treated with 2-(7-Aza-1H-benzotriazole-1-yl)-1,1,3,3-tetramethyluronium hexafluorophosphate (HATU) (13.45 g, 35.3 mmol). The reaction mixture was stirred for 12 hours at room temperature, quenched with water, and then extracted with ethyl acetate (EtOAc) (3×200 mL). The combined organic layers were washed with HCl (aqueous 1N, 200 mL), dried over sodium sulfate (Na₂SO₄) and concentrated to yield a yellow solid. The crude product was washed with a minimum amount of dichloromethane to yield the title compound (8.5 g, 65% yield) as a white solid. M/Z 440.

### b. (R)—N-[(4-chlorophenyl)sulfonyl]phenylalanine

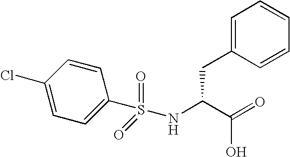

To a suspension of D-phenylalanine (100 g, 606 mmol) in a solution of sodium bicarbonate (103 g, 1.2 mol) in water was added p-chlorophenylsulfonyl chloride (140.6 g, 666 mmol). After stirring overnight at room temperature, the reaction mixture was filtered and washed with dichloromethane, and then was acidified with HCl (6N) and extracted with EtOAc. The combined organic extracts were dried, filtered, and concentrated to give 109 g (53% yield) of the title compound. M/Z 339.

The following Reference Compounds and Intermediates were prepared in a similar manner:

### A.2 (R)-4-Chloro-N-[1-(5-mercapto-4-methyl-4H-1,2,4-triazol-3-yl)-2-phenylethyl]benzenesulfonamide M/Z 408

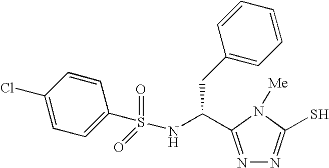

### A.3 N-[(1R)-1-(4-ethyl-5-mercapto-4H-1,2,4-triazol-3-yl)-2-phenylethyl]-4-fluorobenzenesulfonamide

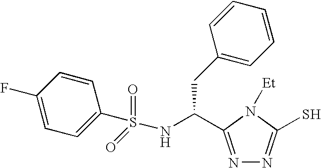

### A.4-chloro-N-[(1R)-1-(4-ethyl-5-mercapto-4H-1,2,4-triazol-3-yl)ethyl]benzenesulfonamide

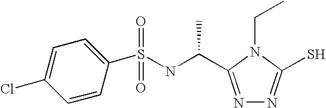

### A.5 N-[(1R)-1-(4-ethyl-5-mercapto-4H-1,2,4-triazol-3-yl)ethyl]-4-fluorobenzenesulfonamide

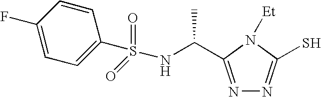

### A.6 4-chloro-N-[1-(4-ethyl-5-mercapto-4H-1,2,4-triazol-3-yl)-2-methylpropyl]benzenesulfonamide

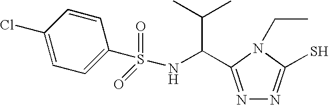

### Preparation of 4-chloro-N-(1R)-1-{5-[(cyclopropylmethyl)thio]-4-methyl-4H-1,2,4-triazol-3-yl}-2-phenylethyl)benzenesulfonamide

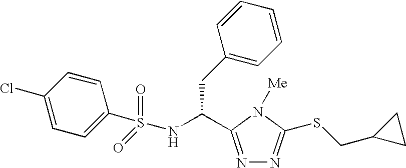

To a stirred suspension of cesium carbonate (500 mg, 1.5 mmol) and 4-chloro-N-[(1R)-1-(4-methyl-5-thioxo-4,5-dihydro-1H-1,2,4-triazol-3-yl)-2-phenylethyl]benzenesulfonamide (Intermediate A.2, 200 mg, 0.47 mmol) in tetrahydrofuran (THF) (5 mL) under a nitrogen atmosphere was added bromomethylcyclopropane (50 μL, 0.52 mmol). The mixture was stirred for 2 hours at room temperature, after which time the reaction was judged complete by LC-MS. Excess thiophenol scavenger resin was added to remove excess alkylating agent and the mixture was agitated for 2 hours followed by filtration. The filtrate was diluted with water and extracted with ethyl acetate (2×50 mL), dried (Na₂SO₄), filtered, and subjected to Super Critical Fluid (SCF) chromatography (Berger Diol, 20×250 mm column, eluent 15% methanol in CO₂, monitored at 220 nm). Like fractions were combined to give 34 mg (0.064 mmol, 14% yield) of the title compound. ¹H NMR (400 MHz, MeOD) 67.57 (d, J=7.58 Hz, 2H), 7.38 (d, J=7.83 Hz, 2H), 7.14 (s, 3H), 7.03 (s, 2H), 4.74 (t, J=7.83 Hz, 1H), 3.24 (s, 3H) 3.34 (s, 2H), 3.22-3.11 (m, 2H), 2.94-2.83 (m, 2H), 0.97-0.87 (m, 1H), 0.51 (d, J=7.83 Hz, 2H), 0.16 (d, J=3.54 Hz, 2H).

M/Z 463.

The following reference compounds were prepared in a similar manner.

| | | | | | Alkylating |
|---|---|---|---|---|---|
| Ex. | Reference Compound | ¹H NMR* | m/z | SM | agent |
| 1.2 | 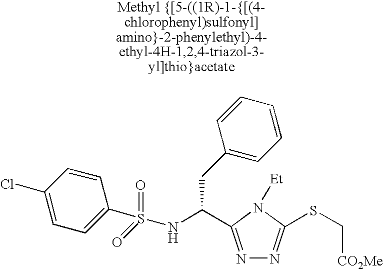 | 7.49(d, J = 8.59 Hz, 2H), 7.33(d, J = 8.59 Hz, 2H), 7.10(s, 1H), 7.09(d, J = 2.27 Hz, 2H), 7.01- 6.92(m, 2H), 4.66(s, 1H), 3.80(s, 2H), 3.62(s, 3H), 3.24-3.21(m, 4H), 3.09(d, J = 7.83 Hz, 2H) | 495 | A.1 | Methylbromo acetate |
| 1.3 | 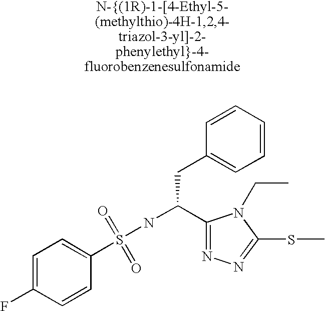 | 1H NMR (300 MHz, DMSO-D6) 0.86(t, J = 7.16 Hz, 3H), 2.51- 2.56(m, 3H), 3.02(d, J = 6.78 Hz, 1H), 3.18(d, J = 8.67 Hz, 1H), 3.65(dd, J = 10.17, 7.35 Hz, 2H), 4.57(d, J = 6.97 Hz, 1H), 7.03-7.12(m, 2H), 7.14-7.22(m, 3H), 7.28(t, J = 8.85 Hz, 2H), 7.60-7.67(m, 2H), 8.85(d, J = 8.48 Hz, 1H) | 421 | A.3 | Methyl iodide |
| 1.4 | 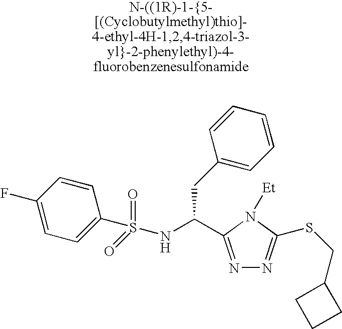 | Not available | 474 | A.3 | Bromomethylcyclo- butane |
| 1.5 | 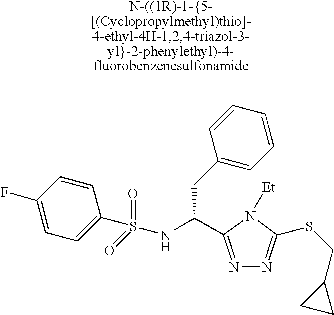 | Not available | 460 | A.3 | Bromomethylcyclo- propane |
| 1.6 | 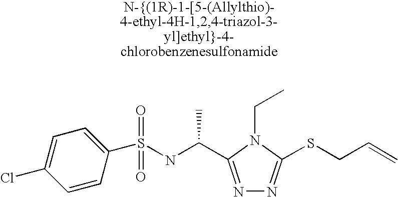 | 1H NMR (300 MHz, DMSO-D6) ppm 1.21 (t, J = 7.16 Hz, 3H), 1.26 (d, J = 6.78 Hz, 2H), 3.79 (d, J = 6.97 Hz, 2H), 3.93 (td, J = 14.46, 7.25 Hz, 2H), 4.70(ddd, J = 15.26, 6.97, 6.78 Hz, 1H), 5.10 (d, J = 9.98 Hz, 2H), 5.23 (dd, J = 16.95, 1.32 Hz, 1H), 5.85-5.99(m, 1H), 7.63-7.69(m, 2H), 7.74-7.81(m, 2H), 8.56(d, J = 8.48 Hz, 1H) | 386 | A.4 | Allyl bromide |
| 1.7 | 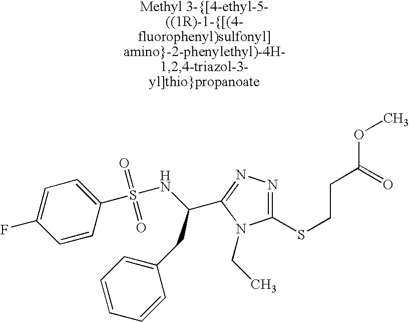 | Not available | 492 | A.3 | Methylbromo acetate |
| 1.8 | 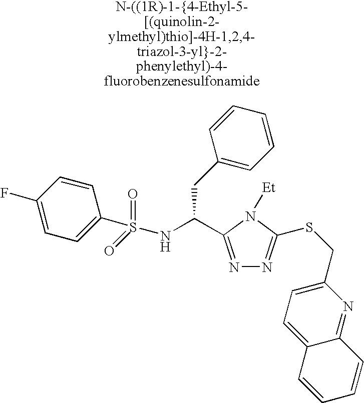 | Not available | 547 | A.3 | 2- (Chloromethyl) quinoline |
| 1.9 | 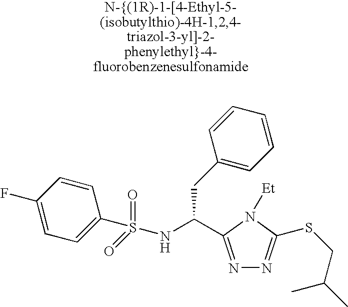 | Not available | 462 | A.3 | 1-Chloro-2- methyl propane |
| 1.10 | 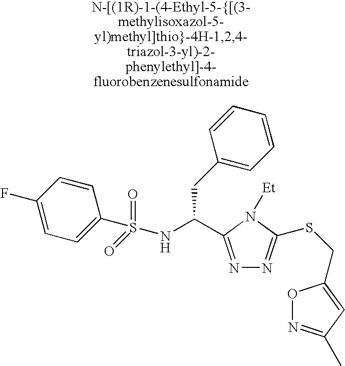 | Not available | 501 | A.3 | 5- (Chloromethyl)- 3- methylisoxazole |
| 1.11 | 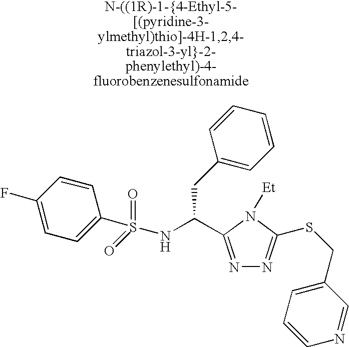 | 300 MHz, MeOD 8.44 (s, 1H), 8.36(d, 1H), 7.60-7.70(m, 3H), 7.30(dd, 1H), 7.02- 7.10(m, 5H), 6.85- 6.90(m, 2H), 4.51(dd, 1H), 4.21-4.34(m, 2H), 3.41-3.67(m, 2H), 3.11-3.19(m, 1H), 2.93-3.01(m, 1H), 0.60-0.67(m, 3H) | 497 | A.3 | 3-(Chloromethyl) pyridine |
| 1.12 | 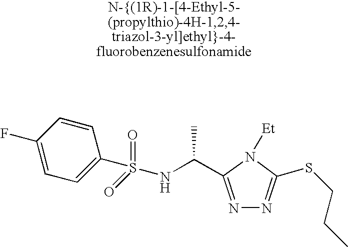 | Not Available | 372 | A.5 | 1-Bromopropane |
| 1.13 | 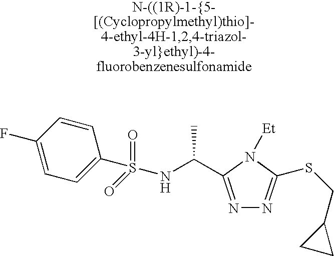 | Not Available | 384 | A.5 | Bromomethylcyclo- propane |
| 1.14 | 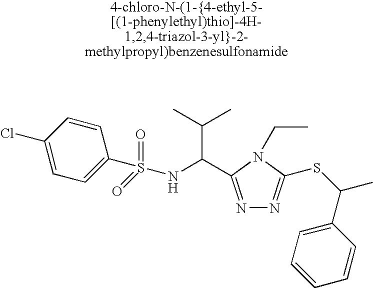 | 8.64-8.66(d, 1H), 7.56- 7.61(m, 2H), 7.47- 7.52(m, 2H), 7.21- 7.31(m, 5H), 4.57-4.68 (m, 1H), 4.06-4.14(m, 1H), 3.58-3.68(m, 2H), 2.00-2.06(m, 1H), 1.61-1.64(dd, 3H), 0.95 (ddd, 3H), 0.89(dd, 3H), 0.59-0.64(t, 3H) | 479 | A.6 | (1-Chloroethyl) benzene |
| 1.15 | 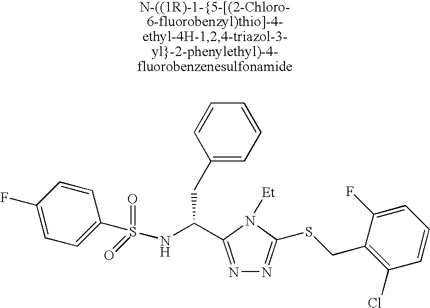 | 300 MHz, MeOD 7.73 (dd, 2H), 7.24-7.35 (m, 2H), 7.14-7.23(m, 5H), 6.97-7.09(m, 3H), 4.63(dd, 1H), 4.29- 4.41(m, 2H), 3.62- 3.78(m, 2H), 3.25(d, 1H), 3.03(dd, 1H), 0.76 (t, 3H) | 549 | A.3 | 1-Chloro-2- (chloromethyl)- 3-fluorobenzene |
| 1.16 | 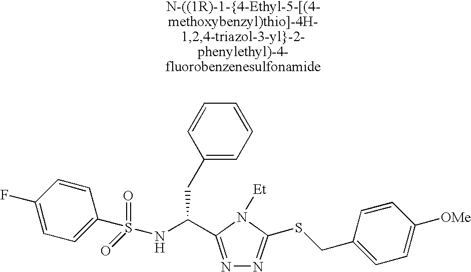 | Not available | 526 | A.3 | p- Methoxybenzyl chloride |
| 1.17 | 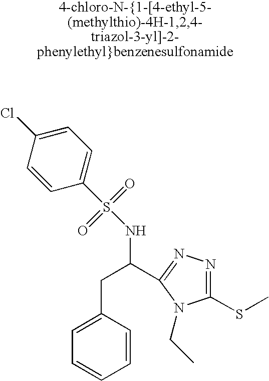 | ¹H NMR (300 MHz, DMSO-d6) 7.52(4H, m), 7.16(3H, m), 7.07 (2H, m), 4.57(1H, m), 3.63(2H, m), 3.18(1H, dd), 3.11(1H, dd), 2.55 (3H, s), 0.85(3H, t). | 436 | A.6 | Methyl iodide |

*Unless otherwise indicated, 400 MHz, MeOD measured as δ.

### B. Synthesis of Non-Mercapto Intermediates

The following compounds were synthesized in a similar manner as described above.

### Example 4

The following Reference compounds were synthesized according to the same procedure.

| | | | | | Alkoxide |
|---|---|---|---|---|---|
| Ex. | Reference Compound | ¹H NMR* | m/z | SM | reagent |
| 9.2 | 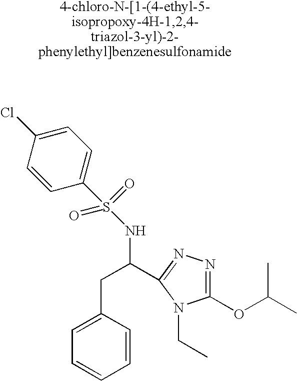 | (300 MHz, CDCl₃) 8.87 (1H, d), 7.60(4H, m), 7.24(5H, m), 4.93(1H, m), 4.54(1H, m), 3.52 (1H, m), 3.22(1H, dd), 3.18(1H, dd), 1.35(6H, m), 0.91(3H, t). | 449 | C.1 | Sodium iso- propoxide |
| 9.3 | 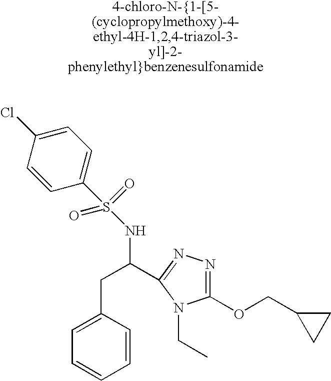 | (300 MHz, CDCl₃) 8.78 (1H, d), 7.53(4H, m), 7.12(5H, m), 4.46(1H, m), 4.05(2H, d), 3.48 (2H, d), 3.15(1H, dd), 3.10(1H, dd), 1.23(1H, m), 0.85(2H, t), 0.56 (2H, m), 0.32(2H, m) | 461 | C.1 | Sodium cyclopropylmethox- ide |
| 9.4 | 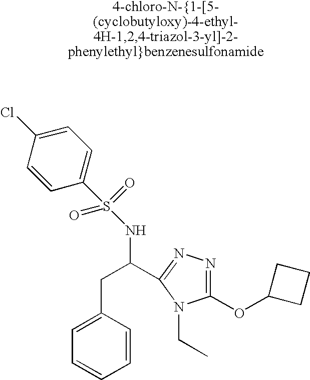 | (300 MHz, CDCl₃) 8.79 (1H, d), 7.53(4H, m), 7.12(5H, m), 4.92(1H, m), 4.45(1H, m), 3.48 (2H, m), 3.12(1H, dd0, 2.96(1H, dd), 2.34(2H, m), 2.01(2H, m), 1.75 (1H, m), 1.59(1H, m), 0.86(3H, t) | 461 | C.1 | Sodium cyclobutyloxide |

### Reference Example 2.1

### Preparation of N-({4-allyl-5-[(cyclopentylmethyl)thio]-4H-1,2,4-triazol-3-yl}methyl)-4-chlorobenzenesulfonamide

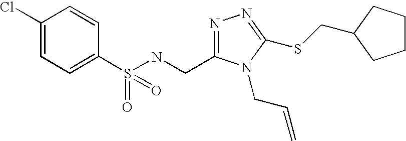

N-[(4-allyl-5-mercapto-4H-1,2,4-triazol-3-yl)methyl]-4-chlorobenzenesulfonamide (bought from Bionet, catalog number 4H-453s, 1 mL of 0.2 M solution in anhydrous DMF, 0.2 mmol) and MP-carbonate resin (Argonaut Technologies, 800269, 3.14 mmol/g, 1.0 mmol) was shaken in a 20 mL scintillation vial for 5 minutes at room temperature. A solution of the alkylating agent (0.5 mL of 0.4 M (iodomethyl)cyclopentane in anhydrous DMF, 0.2 mmol) was added dropwise to the reaction mixture. The mixture was further shaken overnight at room temperature on an orbital shaker. The reaction mixture was transferred off the resin into a Whatman Autovial Filter (0.45 μM), followed by MP-carbonate resin washes of 3×2 mL DMF and 3×2 mL MeOH. The reaction solution and washes were then filtered into a tared 20 mL scintillation vial and dried overnight in a Genevac HTI evaporator at 50° C. The compound was then dissolved in 250 μL DMF, followed by 2 mL MeOH and purified via supercritical fluid chromatography. The purified sample was then evaporated to dryness in a Genevac HTI evaporator at 50° C. to give the title compound 24 mg, (30% yield) as a solid.

¹H NMR (400 MHz, DMSO-D6) ppm 8.46 (t, 1H), 7.71-7.77 (m, 2H), 7.60-7.67 (m, 2H), 5.78-5.89 (m, 1H), 5.18 (dd, 1H), 4.89 (dd, 1H), 4.51 (d, 2H), 4.13 (d, 2H), 3.08 (d, 2H), 2.04-2.16 (m, 1H), 1.68-1.78 (m, 2H), 1.54-1.61 (m, 2H), 1.47-1.53 (m, 2H), 1.17-1.28 (m, 2H); m/z: 426

The following compounds were synthesized according to the same procedure. In the case of alkylation with bromocyclopentane, heating to 50° C. overnight was required.

| | | | | | Alkylating |
|---|---|---|---|---|---|
| Ex. | Reference Compound | ¹H NMR* | m/z | SM | Agent |
| 2.2 | 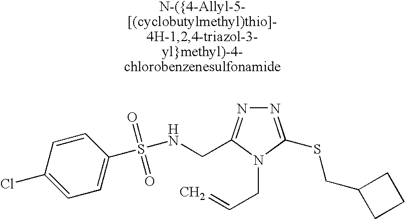 | 8.46(t, 1H), 7.73- 7.76(m, 2H), 7.63- 7.67(m, 2H), 5.83 (ddd, 1H), 5.18(dd, 1H), 4.88(dd, 1H), 4.50(d, 2H), 4.12 (d, 2H), 3.13-3.16 (m, 2H), 2.44-2.55 (m, 1H), 1.96-2.04 (m, 2H), 1.74-1.83 (m, 2H), 1.63-1.71 (m, 2H) | 412 | Bionet 4H-453s | (Bromomethyl) cyclobutane |
| 2.3 | 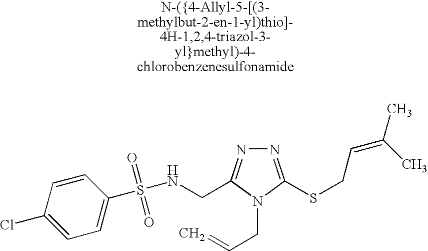 | 8.45(s, 1H), 7.76 (ddd, 2H), 7.66 (ddd, 2H), 5.78- 5.89(m, 1H), 5.25- 5.32(m, 1H), 5.18 (dd, 1H), 4.89(dd, 1H), 4.52(d, 2H), 4.12(d, 2H), 3.69 (d, 2H), 1.66(s, 3H), 1.55(d, 3H) | 412 | Bionet 4H-453s | 1-Chloro-3- methylbut-2- ene |
| 2.4 | 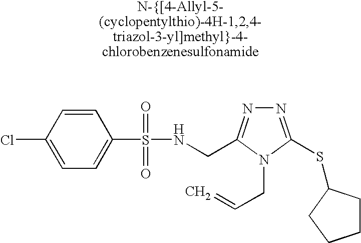 | 8.47(t, 1H), 7.75 (ddd, 2H), 7.65 (ddd, 2H), 5.79- 5.89(m, 1H), 5.17 (dd, 1H), 4.87(dd, 1H), 4.53(d, 2H), 4.13(d, 2H), 3.70- 3.78(m, 1H), 1.96- 2.07(m, 2H), 1.63- 1.72(m, 2H), 1.50- 1.61(m, 4H) | 412 | Bionet 4H-453s | Bromocyclo pentane |
| 2.5 | 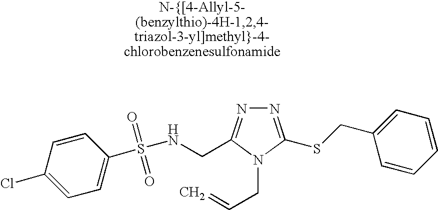 | 8.43(s, 1H), 7.75 (ddd, 2H), 7.65 (ddd, 2H), 7.23- 7.33(m, 5H), 5.67- 5.78(m, 1H), 5.12 (dd, 1H), 4.84(dd, 1H), 4.41(d, 2H), 4.33(s, 2H), 4.11(s, 2H) | 434 | Bionet 4H-453s | (Chloromethyl) benzene |
| 2.6 | 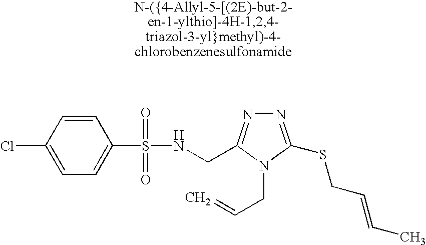 | 8.45(s, 1H), 7.73- 7.78(m, 2H), 7.66 (ddd, 2H), 5.78- 5.89(m, 1H), 5.51- 5.63(m, 2H), 5.18 (dd, 1H), 4.84-4.92 (m, 1H), 4.49-4.59 (m, 2H), 4.12(s, 2H), 3.67(d, 2H), 1.58(ddd, 3H) | 398 | Bionet 4H-453s | (2E)-1-Bromo but-2-ene |
| 2.7 | 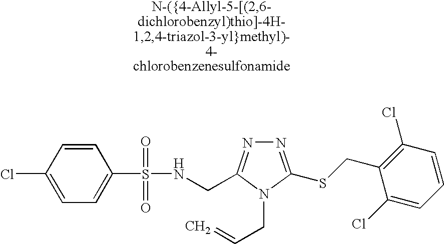 | — | 503 | — | 2- Bromomethyl- 1,3-dichloro- benzene |
| 2.8 | 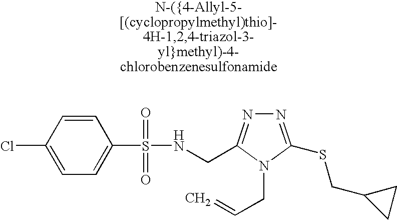 | 8.46(s, 1H), 7.75 (ddd, 2H), 7.65 (ddd, 2H), 5.79- 5.89(m, 1H), 5.19 (dd, 1H), 4.91(dd, 1H), 4.53(d, 2H), 4.13(d, 2H), 3.02 (d, 2H), 1.03-1.13 (m, 1H), 0.51(ddd, 2H), 0.24(ddd, 2H) | 398 | Bionet 4H-453s | (Bromomethyl) cyclopropane |
| 2.9 | 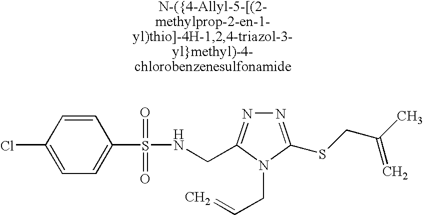 | 8.45(s, 1H), 7.73- 7.77(m, 2H), 7.63- 7.67(m, 2H), 5.80- 5.89(m, 1H), 5.18 (dd, 1H), 4.89(dd, 1H), 4.81(dt, 2H), 4.52-4.56(m, 2H), 4.12(d, 2H), 3.73 (d, 2H), 1.76(s, 3H) | 398 | Bionet 4H-453s | 3-Chloro-2- methylprop-1- ene |
| 2.10 | 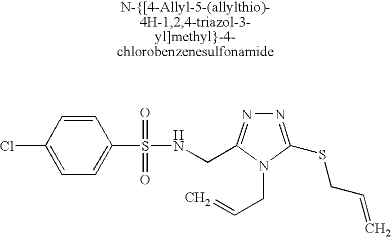 | 8.46(t, 1H), 7.75 (ddd, 2H), 7.66 (ddd, 2H), 5.79- 5.91(m, 2H), 5.20 (d, 1H), 5.17(dd, 1H), 5.07(dd, 1H), 4.89(dd, 1H), 4.53 (d, 2H), 4.13(d, 2H), 3.74(d, 2H) | 384 | Bionet 4H-453s | 3-Chloroprop- 1-ene |
| 2.11 | 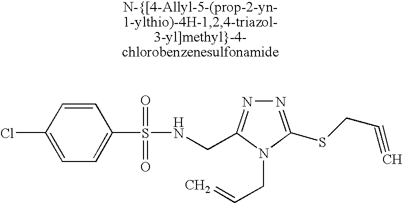 | Not available | 382 | Bionet 4H-453s | 3-Chloroprop- 1-yne |
| 2.12 | 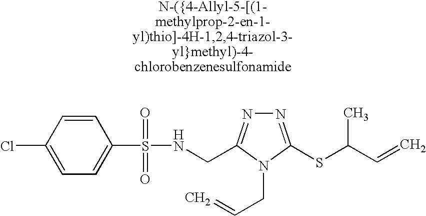 | 8.42-8.50(m, 1H), 7.76(ddd, 2H), 7.66 (ddd, 2H), 5.80- 5.91(m, 2H), 5.17 (dd, 1H), 5.05-5.11 (m, 1H), 4.98-5.04 (m, 1H), 4.87(dd, 1H), 4.57(d, 2H), 4.05-4.15(m, 3H), 1.37(d, 3H) | 398 | Bionet 4H-453s | 3-Chlorobut-1- ene |
| 2.13 | 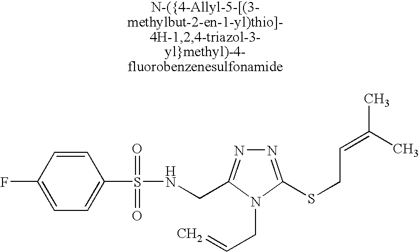 | 8.38(t, 1H), 7.80- 7.86(m, 2H), 7.39- 7.46(m, 2H), 5.78- 5.89(m, 1H), 5.25- 5.31(m, 1H), 5.18 (dd, 1H), 4.89(dd, 1H), 4.52(d, 2H), 4.11(d, 2H), 3.69 (d, 2H), 1.66(s, 3H), 1.55(s, 3H) | 396 | Bionet 8M-902 | 1-Chloro-3- methylbut-2- ene |
| 2.14 | 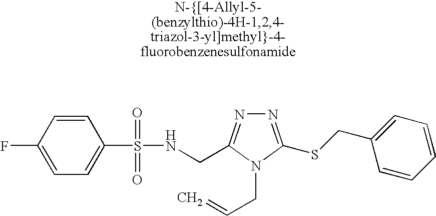 | 8.37(t, 1H), 7.75- 7.85(m, 2H), 7.37- 7.47(m, 2H), 7.23- 7.33(m, 5H), 5.68- 5.78(m, 1H), 5.12 (dd, 1H), 4.84(dd, 1H), 4.41(d, 2H), 4.33(s, 2H), 4.09(d, 2H) | 418 | Bionet 8M-902 | (Chloromethyl) benzene |
| 2.15 | 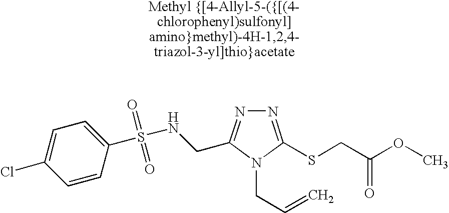 | 8.46(s, 1H), 7.76 (ddd, 2H), 7.67 (ddd, 2H), 5.80- 5.91(m, 1H), 5.22 (dd, 1H), 4.96(dd, 1H), 4.56(d, 2H), 4.13(s, 2H), 4.05(s, 2H), 3.63(s, 3H) | 416 | Bionet 4H-453s | Methyl bromoacetate |
| 2.16 | 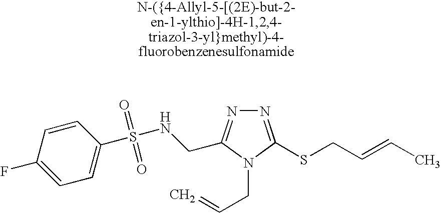 | 8.38(s, 1H), 7.79- 7.85(m, 2H), 7.43 (ddd, 2H), 5.84 (ddd, 1H), 5.49- 5.59(m, 2H), 5.18 (dd, 1H), 4.89(dd, 1H), 4.50-4.59(m, 2H), 4.11(s, 2H), 3.67(dd, 2H), 1.58 (ddd, 3H) | 382 | Bionet 8M-902 | (2E)-1- Bromobut-2- ene |
| 2.17 | 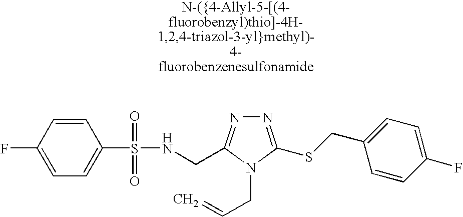 | 8.36(t, 1H), 7.79- 7.85(m, 2H), 7.39- 7.45(m, 2H), 7.35 (tt, 2H), 7.08-7.16 (m, 2H), 5.69-5.80 (m, 1H), 5.12(dd, 1H), 4.83(dd, 1H), 4.42(d, 2H), 4.33(s, 2H), 4.05-4.12(m, 2H) | 436 | Bionet 8M-902 | 1- (Chloromethyl)- 4- fluorobenzene |
| 2.18 | 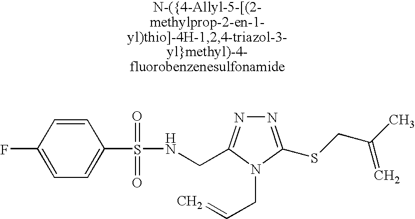 | 8.38(t, 1H), 7.79-7.85(m, 2H), 7.39-7.45(m, 2H), 5.80-5.90(m, 1H), 5.19(dd, 1H), 4.89(dd, 1H), 4.76-4.85(m, 2H), 4.55(d, 2H), 4.11(d, 2H), 3.73(s, 2H), 1.76(s, 3H) | 382 | Bionet 8M-902 | 3-Chloro-2- methylprop-1- ene |
| 2.19 | 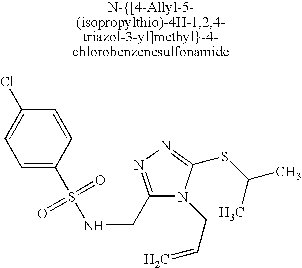 | 8.48(t, 1H), 7.76(ddd, 2H), 7.65(ddd, 2H), 5.79-5.90(m, 1H), 5.17(dd, 1H), 4.87(dd, 1H), 4.52-4.58(m, 2H), 4.14(d, 2H), 3.58(ddd, 1H), 1.28(d, 6H) | 386 | Bionet 4H-453s | 2-bromo propane |
| 2.20 | 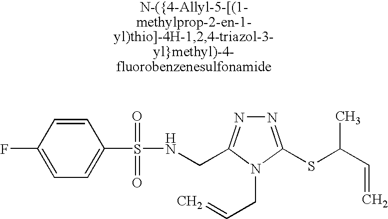 | 8.39(t, 1H), 7.80- 7.85(m, 2H), 7.39- 7.46(m, 2H), 5.80- 5.91(m, 2H), 5.18 (dd, 1H), 5.05-5.12 (m, 1H), 4.98-5.03 (m, 1H), 4.87(dd, 1H), 4.58(d, 2H), 4.05-4.15(m, 3H), 1.37(d, 3H) | 382 | Bionet 8M-902 | 3-chlorobut-1- ene |
| 2.21 | 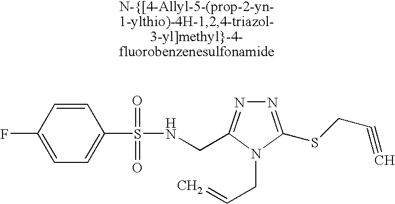 | 8.45(s, 1H), 7.78- 7.84(m, 2H), 7.38- 7.45(m, 2H), 5.80- 5.91(m, 1H), 5.16- 5.23(m, 1H), 4.92 (dd, 1H), 4.59(d, 2H), 4.12(s, 2H), 3.92(d, 2H), 3.22(t, 1H) | 366 | Bionet 8M-902 | 3-Chloroprop- 1-yne |

*1H NMR (400 MHz, DMSO-D6) unless otherwise indicated.

1. A compound of formula (I) 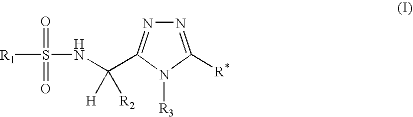
or a pharmaceutically acceptable salt thereof, wherein
R₁ is aryl, heteroaryl, C₁₋₆alkyl, ar(C₁₋₆alkyl), or heteroar(C₁₋₆alkyl) wherein R₁ may be optionally substituted on carbon by one or more substituents selected from C₁₋₃alkyl, halo C₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R₂ is C₁₋₆alkyl, C₂₋₆alkenyl, C₂₋₆alkynyl, C₃₋₆cycloalkyl, C₃₋₆cycloalkyl(C₁₋₆alkyl), aryl, heteroaryl, ar(C₁₋₆alkyl), or heteroar(C₁₋₆alkyl) wherein R₂ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R₃ is C₁₋₆alkyl, C₃₋₆cycloalkyl, C₃₋₆cycloalkyl(C₁₋₆alkyl), heterocyclylC₁₋₆alkyl, ar(C₁₋₆alkyl), C₃₋₆alkenyl, C₃₋₆alkynyl, or heteroar(C₁₋₆alkyl) wherein R₃ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂₅—NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R* is H, C₁₋₆alkyl, C₃₋₆cycloalkyl, aryl, C-linked heteroaryl, C-linked heterocyclyl, C₃₋₆alkenyl, C₃₋₆alkynyl, ar(C₁₋₆alkyl), heteroar(C₁₋₆alkyl), cycloalkyl(C₁₋₆alkyl), heterocyclyl(C₁₋₆alkyl), acyl, C₁₋₆alkoxycarbonyl(C₁₋₆alkyl), cyano or cyanoalkyl wherein R^{*} may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂₅—NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said C-linked heteroaryl, C-linked heterocyclyl, heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N-(C₁₋₆alkyl)carbamoyl, benzyl, benzyloxycarbonyl, benzoyl and phenylsulphonyl;
provided the compound is not N-[1-(4-propyl-4H-1,2,4-triazol-3-yl)ethyl]benzenesulfonamide.

2. A compound of formula (II) 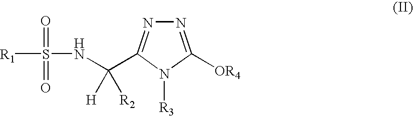
or a pharmaceutically acceptable salt thereof, wherein
R₁ is aryl, heteroaryl, C₁₋₆alkyl, ar(C₁₋₆alkyl), or heteroar(C₁₋₆alkyl) wherein R₁ may be optionally substituted on carbon by one or more substituents selected from C₁₋₃alkyl, halo C₁₋₃alkyl, C₃₋₆cycloalkyl, C₁₋₃alkoxy, C₁₋₃ alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆ alkyl)carbamoyl, benzyl, benzyloxycarbonyl, benzoyl and phenylsulphonyl;
R₂ is heteroar(C₁₋₆alkyl) wherein R₂ may be optionally substituted on carbon by one or more substituents selected from C₁₋₃alkyl, halo C₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R₃ is C₁₋₆alkyl, C₁₋₆cycloalkyl, C₃₋₆cycloalkyl(C₁₋₆alkyl), heterocyclylC₁₋₆alkyl, ar(C₁₋₆alkyl), C₃₋₆alkenyl, C₃₋₆alkynyl, or heteroar(C₁₋₆alkyl) wherein R₃ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heterocyclyl(C₁₋₆alkyl) or heteroar(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆ alkyl)carbamoyl, benzyl, benzyloxycarbonyl, benzoyl and phenylsulphonyl; and
R₄ is H, C₁₋₆alkyl, C₃₋₆cycloalkyl, aryl, heteroaryl, heterocyclyl, C₃₋₆alkenyl, C₃₋₆alkynyl, ar(C₁₋₆alkyl), heteroar(C₁₋₆alkyl), C₃₋₆cycloalkyl(C₁₋₆alkyl), heterocyclyl(C₁₋₆alkyl), acyl, acyloxy, acylamino, C₁₋₆alkoxycarbonyl(C₁₋₆ alkyl), cyano or cyano(C₁₋₆alkyl) wherein R₄ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroaryl, heterocyclyl, heteroar(C₁₋₆alkyl), C₃₋₆cycloalkyl(C₁₋₆alkyl) or heterocyclyl(C₁₋₆alkyl) contain an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl.

3. A compound formula (I) or a pharmaceutically acceptable salt thereof, according to claim 1
wherein
R₁ is aryl wherein aryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring;
R₂ is C₁₋₆alkyl, C₁₋₆cycloalkyl(C₁₋₆alkyl), ar(C₁₋₆alkyl), or heteroar(C₁₋₆ alkyl) wherein R₂ may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring and wherein if said heteroar(C₁₋₆alkyl) contains an —NH— moiety that nitrogen may be optionally substituted by a group selected from C₁₋₆ alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆ alkyl)carbamoyl, benzyl, benzyloxycarbonyl, benzoyl and phenylsulphonyl;
R₃ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring; and
R* is H, C₁₋₆alkyl or C₃₋₆cycloalkyl wherein R* may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂₅—NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

4. A compound of formula (II) or a pharmaceutically acceptable salt thereof, according to claim 2, wherein
R₁ is aryl wherein aryl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃₅ halo, nitro, cyano, ═O, ═S, —OH, —SH, —CF₃, —OCF₃, —CO₂H, —CO₂C₁-C₆alkyl, —NH₂₅—NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring;
R₂ is heteroar(C₁₋₆alkyl) wherein heteroar(C₁₋₆alkyl) may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃₅ halo, nitro, cyano, ═O, ═S, —OH, —SH, —CF₃, —OCF₃, —CO₂H, —CO₂C₁-C₆alkyl, —NH₂₅—NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring wherein if said heteroar(C₁₋₆alkyl) contains an —NH— moiety that nitrogen may be optionally substituted by a group selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R₃ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring; and
R₄ is C₁₋₆alkyl wherein C₁₋₆alkyl may be optionally substituted on carbon by one or more substituents selected from C₁₋₃alkyl, haloC₁₋₃alkyl, C₃₋₆cycloalkyl, C₁₋₃alkoxy, C₁₋₃alkylthio, —O(CH₂)₁₋₅CF₃, halo, nitro, cyano, ═O, ═S, —OH, —SH, —CF₃, —OCF₃, —CO₂H, —CO₂C₁-C₆alkyl, —NH₂, —NH(C₁₋₆alkyl), —CONR′R″, or —N(C₁₋₆alkyl)₂ where R′ and R″ are independently C₁₋₆alkyl or aryl, or together with the nitrogen to which they are attached form a 4- to 7-membered ring.

5. A compound of formula (I) according to claim 1, or a pharmaceutically acceptable salt thereof, selected from
4-Chloro-N-[1-(4-ethyl-5-trifluoromethyl-4H-[1,2,4]triazol-3-yl)-ethyl]benzenesulfonamide;
4-chloro-N-[1-(4,5-diethyl-4H-[1,2,4]triazol-3-yl)-2-(4-fluoro-phenyl)-ethyl]benzenesulfonamide; and
[D]-4-Chloro-N-{1-[4-ethyl-5-(2,2,2-trifluoro-ethyl)-4H-[1,2,4]triazol-3-yl]-ethyl}benzenesulfonamide.

6. A compound of formula (II) according to claim 2, or a pharmaceutically acceptable salt thereof, selected from
4-chloro-N-[1-(4-ethyl-5-methoxy-4H-1,2,4-triazol-3-yl)-2-pyridin-2-ylethyl]benzenesulfonamide;
4-Chloro-N—[(R)-1-(4-ethyl-5-methoxy-4H-[1,2,4]triazol-3-yl)-2-pyridin-3-yl-ethyl]benzenesulfonamide; and
[D]-4-chloro-N-[1-(4-ethyl-5-methoxy-4H-1,2,4-triazol-3-yl)-2-(5-fluoropyridin-2-yl)ethyl]benzenesulfonamide.

7. A pharmaceutical composition which comprises a compound of the formula (I), or a pharmaceutically acceptable salt thereof, as claimed in claim 1 in association with a pharmaceutically-acceptable carrier, diluent or excipient.

8. A pharmaceutical composition which comprises a compound of the formula (II), or a pharmaceutically acceptable salt thereof, as claimed in claim 2 in association with a pharmaceutically-acceptable carrier, diluent or excipient.

9. (canceled)

10. (canceled)

11. (canceled)

12. (canceled)

13. (canceled)

14. (canceled)

15. (canceled)

16. (canceled)

17. A method for producing an Edg-1 antagonistic effect in a warm-blooded animal, such as man, which comprises administering to said animal an effective amount of a compound of formula (I), or a pharmaceutically acceptable salt thereof, as claimed in claim 1.

18. A method for producing an Edg-1 antagonistic effect in a warm-blooded animal, such as man, which comprises administering to said animal an effective amount of a compound of formula (II), or a pharmaceutically acceptable salt thereof, as claimed in claim 2.

19. A method for producing an anti-cancer effect in a warm-blooded animal, such as man, which comprises administering to said animal an effective amount of a compound of formula (I), or a pharmaceutically acceptable salt thereof, as claimed in claim 1.

20. A method for producing an anti-cancer effect in a warm-blooded animal, such as man, which comprises administering to said animal an effective amount of a compound of formula (II), or a pharmaceutically acceptable salt thereof, as claimed in claim 2.

21. A method of treating angiogenesis-related diseases including non-solid tumors, solid tumors and their metastases, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors, in a warm-blooded animal in need of such treatment which comprises administering to said animal an effective amount of a compound of formula (I) or a pharmaceutically acceptable salt thereof, as claimed in claim 1.

22. A method of treating angiogenesis-related diseases including non-solid tumors, solid tumors and their metastases, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors, in a warm-blooded animal in need of such treatment which comprises administering to said animal an effective amount of a compound of formula (II) or a pharmaceutically acceptable salt thereof, as claimed in claim 2.

23. (canceled)

24. (canceled)

25. (canceled)

26. (canceled)

27. Processes for preparing a compound of formula (I) or a pharmaceutically acceptable salt thereof as claimed in claim 1, wherein the variables are, unless otherwise specified, as defined in claim 1, which processes comprise
Process a) reacting a compound of formula (1e) 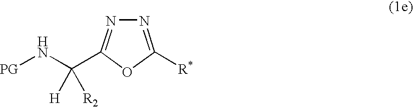
with an amine of the formula (2)
R₃—N₂ (2)
wherein PG is R₁SO₂ or a protecting group, e,g, BOC
and, if PG is a protecting group, further reacting with R₁SO₂Cl
process b) reacting a compound of formula (1e′) 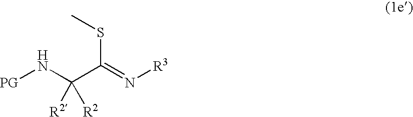
with a hydrazide of formula (2a)
R*—CONHNH₂ (2a)
and, if PG is a protecting group, further reacting with R₁SO₂Cl
and thereafter if necessary:
i) converting a compound of formula (I) into another compound of formula (I);
ii) removing any protecting groups;
iii) forming a pharmaceutically acceptable salt.

28. A process for preparing a compound of formula (II) or a pharmaceutically acceptable salt thereof as claimed in claim 2, wherein the variables are, unless otherwise specified, as defined in claim 2, which process comprises
Process c) reacting a compound of formula (2f) 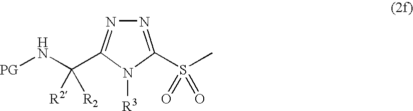
with a compound of the formula (2g)
R⁴ONa (2g)
wherein PG is R₁SO₂
and thereafter if necessary:
i) converting a compound of the formula (II) into another compound of the formula (II);
ii) removing any protecting groups;
iii) forming a pharmaceutically acceptable salt.
